(19) United States
(12) Patent Application Publication
(10) Pub. No.: US 2026/0041857 A1
(43) Pub. Date: Feb. 12, 2026

(54) Negative Pressure Amplification Apparatus and Inhaler

(71) Applicant: Cambridge Healthcare Innovations Limited, Cambridge (GB)

(72) Inventors: David Harris, Cambridge (GB); Warren Isaacs, Cambridge (GB)

(21) Appl. No.: 19/103,498

(22) PCT Filed: Aug. 11, 2023
(86) PCT No.: PCT/GB2023/052131

§ 371 Date: Feb. 12, 2025

(30) Foreign Application Priority Data
Aug. 12, 2022 (GB) 2211840.0
Aug. 12, 2022 (GB) 2211849.1

(51) Int. Cl. A61M 11/02; A61M 15/00

(52) U.S. Cl. CPC A61M 11/02; A61M 15/0005; A61M 15/0021; A61M 2202/064; A61M 2206/16

(57) ABSTRACT

A negative pressure amplifier apparatus comprises a first amplifier chamber comprising a first amplifier inlet, a first primary inlet, and a first outlet. The first outlet forms, or is in fluid connection with, an apparatus outlet. The first amplifier chamber is configured to establish, in response to fluid being drawn from the apparatus outlet, a first primary fluid flow from the first primary inlet to the first outlet, and to create a first reduced-pressure zone of fluid at the first amplifier inlet, such that a first fluid flow is drawn through the first amplifier inlet into the first reduced-pressure zone. The first amplifier inlet is positioned downstream of the first primary inlet, such that in use, the first fluid flow is introduced to the first reduced-pressure zone downstream of the first primary inlet. An inhaler apparatus comprising the negative pressure amplification apparatus is also provided.

The present invention relates to a negative pressure amplification apparatus, and an inhaler comprising the negative pressure amplification apparatus. In particular, the invention relates to a negative pressure amplification apparatus and inhaler for improving the delivery of a medicament to a patient.

### BACKGROUND

All current dry powder inhalers (DPIs) rely solely upon harnessing a proportion of the available energy from the patient's inhalation to do work on the powdered formulation, to break up the powdered medicament (deagglomerate the particles), to produce a fine, respirable aerosol. The vast majority of DPIs deliver formulations that comprise two or more different particle size fractions—the “fine” or “respirable” fraction is the drug, also known as the Active Pharmaceutical Ingredient (API), and the bulk of the formulation is the “carrier” fraction, which is comprised of much larger (coarse) non-respirable particles-usually lactose. This is done for two main reasons:
i) Many drugs that are delivered by inhalation are potent, and only require typically 50 μg to a few hundred μg per dose. This is volumetrically a very small amount, and consequently difficult to meter-either in the factory by specialist filling equipment, or inside the inhaler. Mixing this tiny quantity of API with a much larger quantity of inert and coarse carrier particles, typically lactose, bulks up the volume to be metered and therefore increases both the accuracy and consistency of dose metering;
ii) Respirable particles typically need to be less than 5 μm in aerodynamic diameter to travel down into the lungs and not impact earlier on, upstream in the larger bronchioles. Many therapies require deep lung deposition—some as far as the alveoli—and consequently require even finer respirable particles, often referred to as “extra-fine”, which are typically under 2 μm in aerodynamic diameter. All fine particles, such as these, do not “flow” well, meaning handling them during manufacture is very difficult. A simple example would be to compare icing sugar with granulated sugar-identical material, but with different size fractions. If you try to pour icing sugar from a spoon, it doesn't flow, as the average particle size is only a few tens of micrometers. Conversely, granulated sugar, which has an average particle size of a few hundred micrometers, pours from a spoon very easily. By mixing fine, respirable API with a much coarser carrier fraction of lactose to improve the overall flowability, handling the formulation during manufacture becomes much more straightforward.

However, a well-mixed (homogenous) formulation comprising one or more fine APIs with a coarse carrier fraction is difficult to deagglomerate and aerosolise. Many market leading DPIs only achieve 20 to 30% Fine Particle Fraction (FPF), which is the percentage of drug (of the dose emitted from the inhaler) that is below 5 μm—also known as the “respirable fraction”. The remaining drug is predominantly still attached to the carrier particles, which are usually between 50 μm and 200 μm—i.e. much larger than the API particles. It is worth noting that a typical API particle might be 2 μm in diameter, whereas a typical carrier particle size, e.g. DFE Pharma's Lactohale® 200, is approximately 80 μm. Whilst this difference is only a factor of ˜40× in diameter, it is a difference of approximately 60,000× in terms of mass. The physics governing the behaviour of API particles is very different to that of carrier particles, and is indeed complex for both.

As a patient inhales—from any current DPI that uses a carrier-based formulation—virtually all of the formulation leaves the inhaler and enters the patient, except for a small percentage that may be held up within the inhaler. It is certainly the design intent of DPIs to deliver the entire dose to the patient, and ideally have no formulation—API or carrier fraction—remaining within the inhaler device. As the formulation travels with the airflow into the patient's mouth, the carrier particles have sufficiently high inertia such that they are unable to turn with the airflow towards the back of the patient's mouth, and travel down with the airflow into the trachea. Because they are aerodynamically so large, they instead impact and deposit upon the back of the patient's throat. In fact, most particles with an aerodynamic diameter of over 10 μm will not be able to follow the airflow into the trachea, due to their high inertia, and will consequently impact and deposit on the patient's throat. This means that for many inhalers that are available and used today, the majority of the API (drug) is delivered to the patient's mouth and throat, as it is still attached to the carrier particles, rather than reaching the target site of the lungs. For APIs that are steroids, for example, this topical delivery to the mouth and throat region is highly undesirable, and can lead to unwanted side effects such as candidiasis. This does very little to help the patient using the inhaler to remain compliant and adherent to their therapy. Previous inventions, such as the Conix® DPI (Patent application WO2006061637), have sought to retain the vast majority of the lactose carrier fraction in the inhaler to minimise mouth and throat deposition. The Occoris® DPI (Patent application WO2015082895) is designed to work with fine API particles only, and requires zero carrier particles, thus greatly reducing any mouth and throat deposition, as all the emitted aerosol is sufficiently fine and has sufficiently low inertia, such that it is able to follow the airflow into the trachea and avoid impacting on the back of the patient's throat. However, with both of these inventions, and similar, the patient using them receives negligible feedback in the form of taste, that the inhaler has delivered a dose. This is potentially problematic and even dangerous; as the patient could think that their inhaler has not delivered a dose, and may repeat the inhalation one or more times and risk the possibility of overdosing.

Most DPIs emit a dose that comprises almost all of the API and almost all of the carrier fraction. The entrainment of the dry powder formulation happens very quickly, in the first part of the inhalation (inspiratory manoeuvre), and often all of the powder has left the inhaler within the first few hundred milliseconds. As DPIs by their very nature have resistance to the airflow (airflow resistance), a full inspiratory manoeuvre may last several seconds. One advantage of retaining the carrier fraction within the DPI deagglomeration engine (a reverse-flow, frusto conical cyclone, in the case of the Conix DPI), is that work is done on the formulation to detach and deagglomerate the fine API from the carrier particles throughout the entire duration of the inspiratory manoeuvre. As this time period during which work is done on the formulation is now several seconds rather than a few hundred milliseconds, much more thorough deagglomeration can be achieved, which results in a higher FPF—i.e. more drug reaches the deep lungs of the patient, and less remains to be deposited in the mouth and throat region.

Currently DPIs either emit almost everything, or retain virtually the entire carrier fraction and only emit the fine, aerosolised API (in the case of Conix). What would be advantageous would be a deagglomeration system in which the emission rate and quantity of the carrier fraction could be tuned, to efficiently balance the quantity of emitted carrier fraction required to produce useful (taste) feedback to the user, versus the quantity retained within the deagglomeration system to do sufficient work on the formulation to produce a highly efficient FPF.

A huge challenge for all current DPIs is that because they are solely reliant upon harnessing energy from the patient's inspiratory manoeuvre, it is very difficult to achieve consistent delivery of drug when the energy available varies considerably from patient to patient. All currently available DPIs are “passive”, in that they do not have their own energy source, unlike pressurised metered dose inhalers (pMDIs), which contain hydrofluoroalkane (HFA) propellant to produce a droplet aerosol by flash evaporation through a spray orifice (similar to hairspray or other spray cans, only metered). Whilst “active” DPIs are, and have been, in development, right now there are none commercially available. Active DPIs, such as Occoris, overcome the huge variability from one user to another by containing an internal energy source to produce a respirable aerosol, which is independent of the user, or more specifically, it is independent of how the user inhales. There are probably no active DPIs available because they are so complicated to design, optimise and produce. The ideal DPI system comprising a deagglomeration engine, or deagglomeration apparatus, is a system that consistently produces a high fine particle fraction, independently of how the user inhales, and is simple and cost effective to manufacture. It is also small in size and a platform technology that can be used equally across a range of different inhaler types. For example, it could be incorporated into a single-use inhaler, that is discarded after the delivery of one single dose, e.g. for the delivery of vaccines. It could be incorporated into a single-dose reusable inhaler, e.g. for the delivery of non-routine therapies such as pain relief, insulin for diabetes management, etc. It could also be incorporated into a multi unit-dose inhaler, which may contain 30 to 60 individual, pre-metered doses, and is suitable for delivering routine maintenance medication for the treatment of asthma and COPD, on a once or twice daily regimen over a period of a month, for example. The advantage of using a core engine that is a platform across different device embodiments is that the performance remains identical across them all.

There are therefore a number of problems in the prior art relating to DPIs, including; to provide a simple, low-cost core platform technology that is suitable for incorporation into a wide range of dry powder inhalers (DPIs) and is suitable for incorporating an adaptive classification system that:
i) Produces a high and consistent fine particle fraction (FPF) which is independent of how strongly the user inhales; and
ii) Enables the rate of carrier fraction emission from the formulation unit container (e.g. a coldform/foil blister) to be easily tuned.

To improve on conventional DPIs, the primary aim of this adaptive classification system would be to emit only fully deagglomerated, fine API particles. A secondary aim would be that any coarse lactose (or other) carrier particles which might be emitted will ideally have been completely stripped of API particles. This may be achievable by tuning the system to delay the emission of carrier particles towards the end of the inspiratory manoeuvre-such emission being governed by an influenceable probabilistic function. Delaying carrier particle emission facilitates more complete deagglomeration, by extending the time over which work can be done on the formulation, resulting in higher and more consistent FPF. This also means that any carrier particles emitted are more likely to be free of API particles, thus minimising API deposition in the mouth and throat. It further means that the patient may only tend to taste the carrier particles towards the end of the inspiratory manoeuvre, which may make this an effective indication that dose delivery is complete.

### Background Information on Inspiratory Energy:

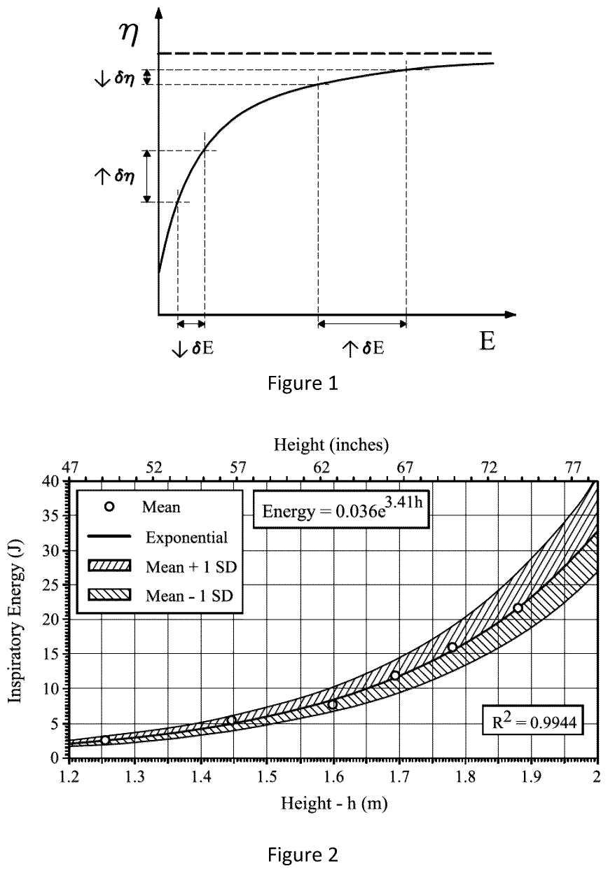

If you examine the relationship between the energy put into a formulation (powder) during inhalation and the efficiency of a deagglomeration engine—i.e. energy harnessed from the inspiratory manoeuvre of the patient and input to the deagglomeration and aerosolisation of the formulation, and the effectiveness of that deagglomeration and aerosolisation of the formulation—there are a few key observations to note, FIG. 1 illustrates the effect of input energy (energy applied to the powder) (x-axis) on the aerosolisation (fine particle) efficiency (y-axis) of typical passive DPIs. Firstly, with almost zero input energy, the most loosely bound API particles will readily detach from their carrier particles simply by being entrained in the airflow, giving rise to the “free” part of the fine particle fraction. The earliest DPIs, such as the Aerohalor® (Abbott Laboratories, 1947), transferred very little energy into the powdered formulation, and consequently only achieved this “free” part of the fine particle fraction—of the order 10-15%, indicated as the intersection with the y-axis, FIG. 1.

Secondly, with current DPIs (e.g. Turbuhaler®, of AstraZeneca, NEXThaler® of Chiesi, Genuiair® of AstraZeneca, Onbrez®, of Novartis, etc.) a higher proportion of the energy available from the patient's inspiratory manoeuvre is transferred to the formulation, which results in greater efficiency and a higher fine particle fraction, FIG. 1. However, it is important to note that, using airflow alone, it is impossible to ever achieve 100% efficiency, as the most tightly bound API particles that are directly on the surface of carrier particles, or even mechanically locked in place in microscopic cracks or imperfections on the carrier particle's surface, cannot be removed without breaking down the close range forces of adhesion—e.g. they would need to be washed off with liquid or dissolved using solvent. Aerodynamic deagglomeration is a probabilistic system—every time a carrier particle collides with another, or with a wall within the deagglomeration engine, for example, there is a chance that API particles will become detached. By the very nature of a probabilistic system, achieving a 100% is infinitely unlikely, hence the efficiency curve asymptotes towards 100%, FIG. 1.

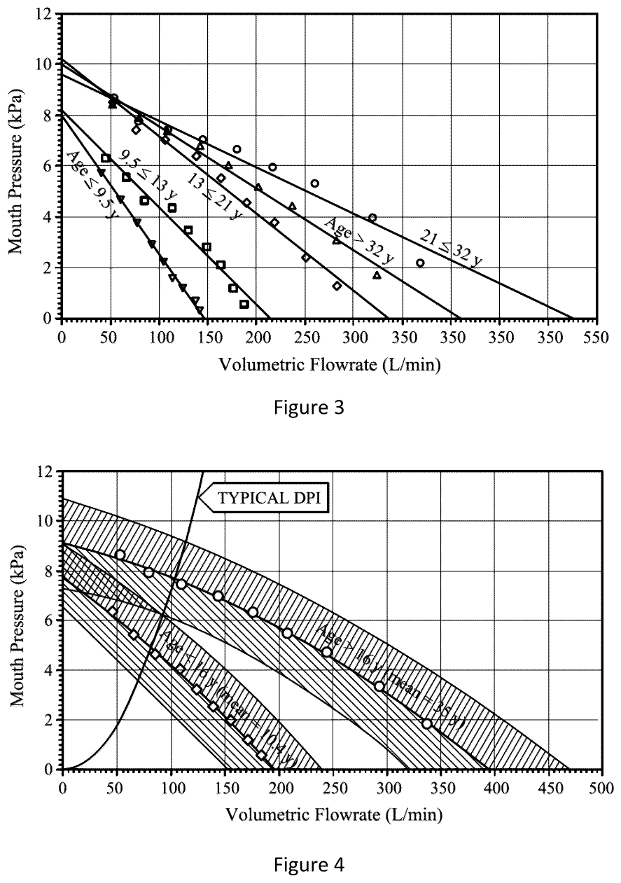

This efficiency curve can be considered as two parts-a steep part and a flatter part. All current DPIs perform (with typical carrier based formulations, at least) on the steep part of this curve, and the “knee” also known as the point where the curve visibly bends, specifically from the steep part to the flatter part, is located at approximately 60-70%. This is a particularly suboptimal operating region, as even a small difference in the input energy results in a large difference in the efficiency. This is evident in real use with the majority of DPIs: Patients who inhale less forcefully put less energy into the formulation and consequently receive a lower fine particle dose than patients who inhale strongly, who put greater energy into the formulation and receive a higher fine particle dose. As one of the primary aims of any respiratory drug delivery system is to deliver a known quantity of drug, independently of how the patient inhales-operating before the knee, on the steepest part of the Energy-Efficiency curve—is unfortunately the worst place to be. It is one of the main reasons why so few DPIs make it to market, as they struggle to make it through clinical studies due to variability in delivered dose, resulting from the wide range of input energy provided by the patient group in the study. It has been shown that the total available inspiratory energy correlates well with a patient's height, irrespective of age or gender, FIG. 2 (Harris, D. S., Scott, N., Willoughby, A., How does airflow resistance affect inspiratory characteristics as a child grows into an adult?, DDL21 Conference Proceedings, 79-87 (December 2010)).

The data shown in FIG. 2 are for healthy volunteers, and yet there is still a ten-fold difference in the average inspiratory energy within the ˜90 volunteers, ranging from ˜3 J to over 30 J. Patients with severe asthma or late stage COPD will extend this range of inspiratory energy considerably. This is a key reason why designing DPIs, that are powered by the patient's lungs, is so challenging, particularly achieving consistent delivery efficiency with such a broad range of input energy.

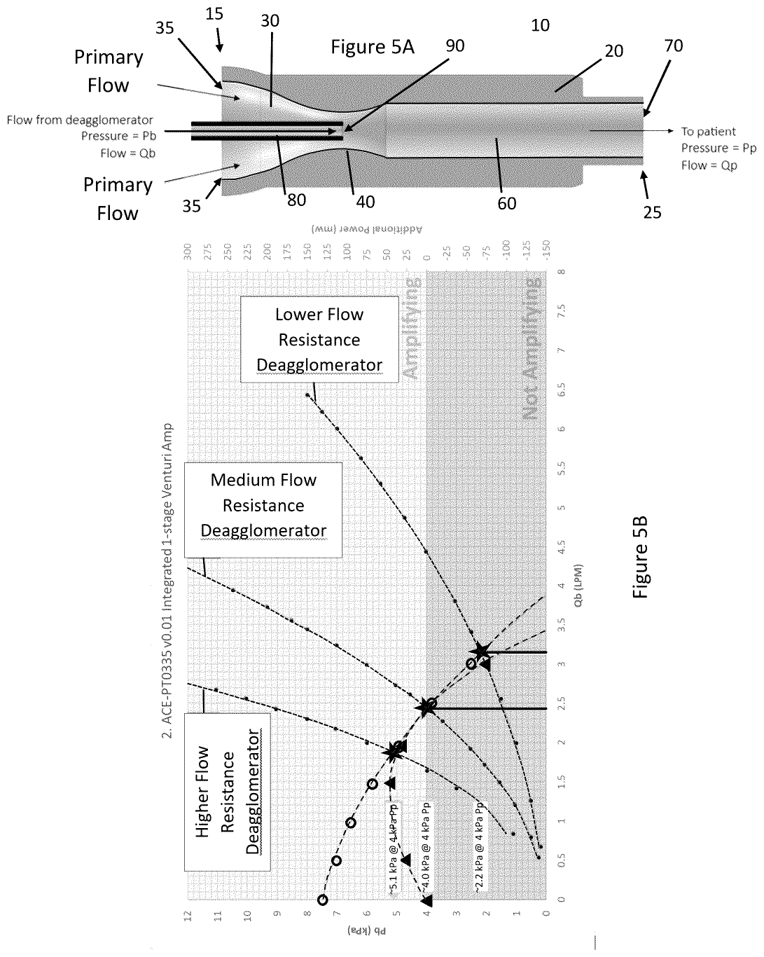

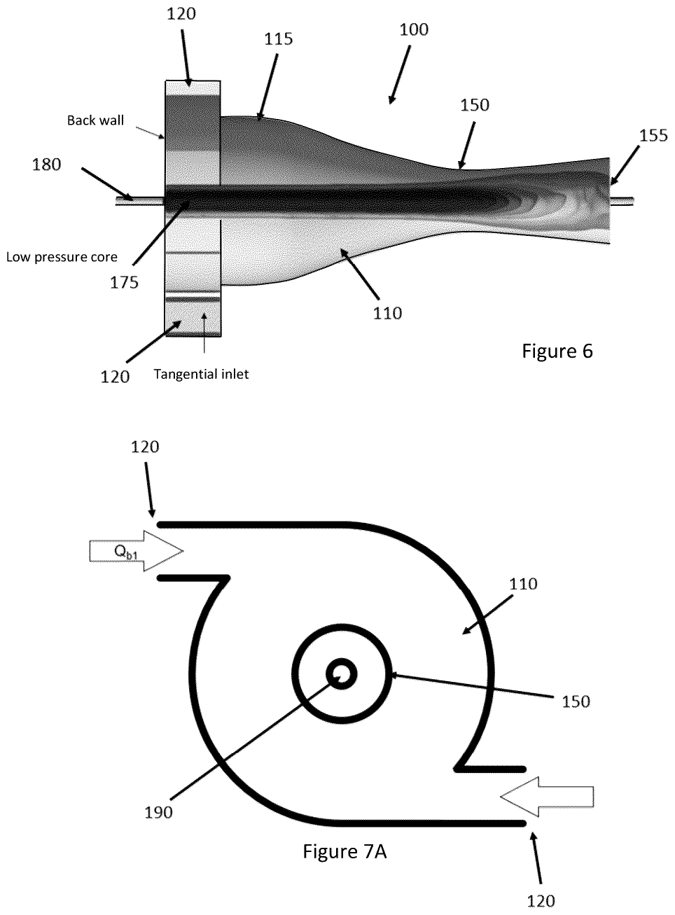

Looking at the Flow-Pressure relationship across range of airflow resistances within this same study, it is clear that there is much greater variation in peak inspiratory flowrate (PIFR) than there is in Mouth Pressure as resistance decreases, FIG. 3 (Harris, D. S., Scott, N., Willoughby, A., How does airflow resistance affect inspiratory characteristics as a child grows into an adult?, DDL21 Conference Proceedings, 79-87 (December 2010)). Again, this will be exacerbated with the inclusion of asthmatic and COPD patients. What these data do suggest is that it is advantageous to design higher resistance DPIs, as they will operate closer towards the left of the graph in FIG. 3, where there is greater consistency between all users. This is illustrated in FIG. 4 (Harris, D. S., Scott, N., Willoughby, A., How does airflow resistance affect inspiratory characteristics as a child grows into an adult?, DDL21 Conference Proceedings, 79-87 (December 2010)).

Another important observation from these results is that healthy volunteers are likely to create a pressure drop across a medium resistance DPI of approximately 4 kPa to 8 kPa. If, however, the resistance of the DPI were higher, it would be reasonable to expect a smaller pressure range, at higher pressures, of approximately 6 kPa to 9 kPa.

### SUMMARY OF INVENTION

The invention provides a negative pressure amplification apparatus and an inhaler as defined in the appended independent claims, to which reference should now be made. Preferred or advantageous features of the invention are set out in dependent subclaims.

In a first aspect of the present invention there is provided a negative pressure amplification apparatus comprising a first amplifier chamber which comprises a first amplifier inlet, a first primary inlet, and a first outlet. The first outlet forms, or is in fluid connection with, an apparatus outlet. The first amplifier chamber is configured to establish, in response to fluid being drawn from the apparatus outlet, a first primary fluid flow from the first primary inlet to the first outlet, and to create a first reduced-pressure zone of fluid at the first amplifier inlet. In use, the first reduced-pressure zone draws a first fluid flow through the first amplifier inlet into the first amplifier chamber. The first amplifier inlet is preferably positioned downstream of the first primary inlet, such that the first fluid flow is introduced to the first reduced-pressure zone downstream of the first primary inlet.

When a negative pressure is applied at the apparatus outlet, fluid is drawn out of the apparatus outlet, and the amplifier apparatus creates the first reduced-pressure zone inside the first amplifier chamber. In the first reduced-pressure zone, the pressure is reduced (more negative) relative to the pressure at the apparatus outlet. The magnitude of the negative pressure in the first reduced-pressure zone is greater than the magnitude of the negative pressure applied at the apparatus outlet, such that the negative pressure is “amplified” inside the first amplifier chamber. The large negative pressure in the first-reduced pressure zone acts to draw the first fluid flow in through the first amplifier inlet, so the first fluid flow is drawn into the first amplifier chamber by a higher negative pressure than is being applied to the apparatus outlet.

The negative pressure amplification apparatus thus advantageously transforms a negative pressure applied to the apparatus outlet into a larger-magnitude negative pressure in the first reduced-pressure zone inside the apparatus. In response to an outlet negative pressure, the apparatus generates a first reduced-pressure zone with a negative pressure greater in magnitude than the outlet negative pressure.

The first amplifier chamber is preferably configured to generate a pressure drop between the first outlet and the first amplifier inlet, such that in response to fluid being drawn from the apparatus outlet by an outlet negative pressure, the first reduced-pressure zone of fluid at the first amplifier inlet experiences a first inlet negative pressure that is greater in magnitude than the outlet negative pressure.

In the present disclosure, for simplicity the invention will be described primarily by reference to air as a preferred fluid. However, the principles of operation are equally applicable to fluids other than air, so embodiments of the invention may be provided in which fluids other than air are drawn through the apparatus.

Likewise, the function of the apparatus will be described primarily in the context of an inhaler, in which air is drawn from the outlet by a patient's inhalation, and the first fluid flow contains an entrained medicament. However, the negative pressure amplification apparatus may advantageously be used in a variety of other applications, and is not limited to the field of inhalers for delivering a medicament to a patient.

In a particularly preferred embodiment, the apparatus forms part of an inhaler for delivering a medicament to a patient, in which a mouthpiece is positioned at the apparatus outlet, the first primary inlet is an air inlet, and the medicament is configured to be entrained in the first fluid flow and drawn into the first amplifier chamber through the first amplifier inlet. In this embodiment, a patient inhales through the mouthpiece, which creates a negative pressure at the apparatus outlet, and draws the first primary flow of air through the apparatus, creating the first reduced-pressure zone at the first amplifier inlet. The negative pressure at the first reduced-pressure zone has a greater magnitude than the patient's inhalation, so the pressure drop across the medicament (between an inlet upstream of the medicament and the first amplifier inlet downstream of the medicament) is amplified. The first fluid flow is drawn in through an apparatus inlet upstream of the medicament, entrains the medicament and is drawn through the first amplifier inlet by a higher negative pressure than can be naturally generated by the patient's inspiratory energy. The first fluid flow (containing the medicament) and the first primary flow then combine and are drawn out of the first outlet to be inhaled by the patient.

The negative pressure amplification apparatus may advantageously transform the energy available from a patient's inspiratory manoeuvre (inhalation), which typically has a high flowrate and a low magnitude negative pressure, into a much higher magnitude negative pressure, albeit at a much lower flowrate.

It is advantageous to have a higher pressure across the medicament than the patient would typically provide, because the maximum velocity of the airflow that can be achieved in the deagglomeration engine is directly related to the pressure drop that is available.

The kinetic energy of the airflow is proportional to the peak velocity squared. Higher kinetic energy (and power) in the airflow is able to do more effective work on the powder formulation and deagglomerate it.

For the typical masses of powder formulation that require deagglomerating and aerosolising, there is too much mass flow of air available from a patient, but at a very limited pressure drop. Trading mass flowrate for a significant increase in pressure drop means that the energy in the airflow through the deagglomeration engine can be much more effective.

Amplifying the pressure drop is particularly useful for patients who have weaker than normal diaphragm muscle strength. These patients cannot work many dry powder inhalers, as they often cannot trigger the breath actuation mechanism that releases the powder automatically upon inhalation, or they may not be able to produce the required pressure drop to aerosolise and deagglomerate the medicament (drug formulation) sufficiently. By amplifying the pressure drop produced by weaker patients, e.g. with a factor of at least 2×, as far as the powder is concerned there is now a patient with healthy lung function inhaling through the device. This makes DPIs incorporating pressure amplification technology much more inclusive of wide patient groups.

Amplifying the negative pressure used to entrain and deagglomerate medicament is highly advantageous, as typical quantities of formulation (powder) in DPIs are very small—usually up to 10 mg per dose. Most conventional DPIs have quite low airflow resistance, which results in people inhaling through them at 30 to 150 LPM (litres per minute)—depending on the inhaler. Even with the highest resistance DPIs, such as Boehringer Ingelheim's HandiHaler®, many users will inhale at least 30 LPM. A litre of air weighs ˜1.2 g (at standard temperature and pressure), so even at the lowest flowrates achieved through current DPIs, this equates to ˜0.5 L/s, or 600 mg/s in terms of the mass flowrate. The point to note is that even this low airflow rate is considerably more massive than the quantity of API (and carrier) that needs to be deagglomerated and aerosolised. The invention may enable trading this unnecessarily high flowrate for an increase in the magnitude of negative pressure. In this way, it may be possible to achieve much higher performance by increasing the effectiveness of the energy transfer into the powder formulation. This is because the airflow velocities that can be reached (e.g. within a deagglomeration engine) directly result from the pressure drop achieved, in accordance with Bernoulli. Moreover, the kinetic energy of the airflow is proportional to the square of the airflow velocity-so doubling the airflow velocity results in four times the kinetic energy. This is important for any DPI design, as it is the kinetic energy available in the airflow that may do work on the dry powder formulation in order to deagglomerate the particles and create a fine, respirable aerosol.

The transformation of a patient's (typically high flowrate+low pressure drop) inspiratory energy into a more useful (low flowrate+high pressure drop) energy preferably enables the creation of an optimal flow regime within a classification deagglomeration engine, and consequently moves the performance into the flatter region at the right-hand side of the Energy—Efficiency curve (FIG. 1). Operating in this region of the Energy-Efficiency curve achieves two advantageous results:
i) The fine particle fraction is much higher, meaning more drug goes into the deep lung and less drug is deposited in the mouth and throat of the patient, and;
ii) As this part of the curve is flatter, any variation in the strength of the inspiratory manoeuvre between patients results in less variation in the delivered dose, meaning delivered dose uniformity is better.

In inhalers incorporating the present invention, the medicament is delivered into the first reduced-pressure zone entrained in the first fluid flow, rather than delivering the medicament into the primary fluid flow, which has a higher flow rate than the first fluid flow. This is counter to the aims of DPIs in the prior art, as prior art inhalers typically aim to inject powdered medicament formulations into airflows with the flow rates as high as possible, so that the powder is entrained in the fast airflow and deagglomerated by crashing into the chamber walls as the primary flow passes through the device. In the present case, however, the first fluid flow with the entrained powder is introduced into the reduced pressure zone at a much lower flow rate. As is described further below, the first amplifier inlet may even be positioned close to the first outlet so that the medicament-containing first fluid flow is not swirled around the amplifier chamber before passing out of the first outlet. This is very different from the prior art.

A further advantage of the invention is that the negative pressure amplifier apparatus may advantageously transform and normalise the efficiency of deagglomeration using the (variable) input energy available from different users, so that the extent to which the powdered formulation is deagglomerated and aerosolised remains more consistent between different users or patients, even if they are capable of different inhalation pressures and air flow rates.

The apparatus preferably comprises one or more apparatus inlets which are configured in fluid communication with each of the inlets into the amplifier chamber(s). The apparatus may comprise a single apparatus inlet through which fluid can enter the apparatus in response to fluid being drawn out of the apparatus outlet, and ducting configured to direct a first fluid flow through the first amplifier inlet, and a separate first primary fluid flow through the first primary inlet. Alternatively the amplifier inlet(s) may be in fluid communication with an apparatus inlet, and the primary inlet(s) may be in fluid communication with one or more primary apparatus inlets.

The first amplifier inlet is a fluid inlet through which the first fluid flow can enter the first amplifier chamber. The primary inlet is a fluid inlet through which the first primary fluid flow can enter the first amplifier chamber. The inlets are preferably configured so that the first primary fluid flow has a greater volume flow rate than the first fluid flow. As it is the first primary flow which establishes the first reduced-pressure zone, the higher-volume flow through the primary inlet may be considered the “primary”, or “driving” flow of fluid. In order to increase the volume of fluid flow in the first primary fluid flow, the apparatus may comprise a plurality of primary inlets, the plurality of primary inlets preferably being spaced circumferentially around the first amplifier chamber.

In response to a negative pressure at the first outlet (or the apparatus outlet), the first primary fluid flow is drawn into the first amplifier chamber through the first primary inlet, and flows through the first amplifier chamber towards the first outlet. The passage of the first primary fluid flow from the first primary inlet to the first outlet creates the first reduced-pressure zone at the first amplifier inlet. The negative pressure at the first reduced-pressure zone draws the first fluid flow into the first amplifier chamber through the first amplifier inlet. Both the first primary fluid flow and the first fluid flow are drawn out of the first outlet by the negative pressure applied at the first outlet.

The first amplifier chamber may be configured to create the first reduced-pressure zone by forming the first primary fluid flow as a cyclonic flow from the first primary inlet to the first outlet, so that the first reduced-pressure zone is formed by a low-pressure core at the centre of the cyclonic flow. Alternatively the first amplifier chamber may be configured to create the first reduced-pressure zone as a result of the venturi effect, by constricting the cross-section of the first amplifier chamber between the first primary inlet and the first outlet. These embodiments are discussed further below.

The first amplifier chamber comprises a conduit having an inlet end and an outlet end. The first outlet is positioned at the outlet end, and the first primary inlet is preferably positioned at or near the inlet end of the first amplifier chamber.

Fluid flow through the first amplifier chamber is directed from the inlet end towards the outlet end in response to a negative pressure at the outlet. Hence the inlet end is the upstream end of the chamber, and the outlet end is the downstream end of the chamber.

The chamber is preferably tapered from a larger diameter at the inlet end to a smaller diameter at the outlet end, and comprises a wall extending from the inlet end to the outlet end. Particularly preferably the first amplifier chamber may be frusto-conical, with the first outlet formed by an aperture at the narrow end of the frusto-conical first amplifier chamber. The first outlet is preferably formed by the narrowest portion of frusto-conical first amplifier chamber. The apparatus may comprise a further passage downstream of the narrowest portion of the frusto-conical first amplifier chamber, but the first amplifier chamber is considered to end at the narrowest portion of the conduit. The first outlet may be formed by a narrowed neck at the downstream end of the first amplifier chamber.

In a preferred embodiment, the apparatus comprises an outlet passage downstream of the first outlet, the outlet passage widening from the first outlet to an apparatus outlet with a diameter greater than the first outlet. The outlet passage may be frusto-conical in shape, and the direction of the outlet passage may preferably be inverted relative to the first amplifier chamber such that the frusto-conical outlet passage is oriented with its narrow end at the first outlet and its wide end at the apparatus outlet.

The first amplifier chamber is preferably symmetrical around a central longitudinal axis. The longitudinal axis may extend along the centre of the frusto-conical chamber from the inlet end to the outlet end of the chamber.

The first amplifier housing is preferably configured so that the first reduced-pressure zone is created on the central axis in response to the first primary fluid flow flowing from the first primary inlet to the first outlet.

The first amplifier inlet is preferably positioned on the central axis of the first amplifier housing. The position of the first amplifier inlet along the central axis of the amplifier chamber may vary, which in turn varies the relative separations between the first amplifier inlet and each of the primary inlet, the tapering walls of the amplifier chamber, and the first outlet. Varying the position of the first amplifier inlet relative to the primary inlet, the chamber walls and the first outlet varies the position of the first amplifier inlet in the first reduced-pressure zone.

At least a portion of the primary inlet is preferably upstream of the first primary inlet. This may advantageously ensure that the primary fluid flow is established upstream of the first primary inlet, so that the first inlet is positioned in the first reduced-pressure zone.

The first amplifier inlet is preferably at least coincident but preferably downstream of the primary inlet's point of entry to the amplifier chamber (the point where fluid enters the amplifier chamber from the primary inlet). As a primary inlet could in some designs comprise a lengthened passage or conduit between the point where the fluid enters, and the point where the fluid exits the passage and enters the amplifier chamber, the position of the primary inlet is defined as its point of entry into the amplifier chamber. Likewise, the position of the amplifier inlet is defined as its point of entry into the amplifier chamber.

The first amplifier inlet is preferably advantageously positioned downstream of the first primary inlet. As the primary inlet typically extends over a short longitudinal distance, the first amplifier inlet is preferably positioned downstream of the downstream-edge of the first primary inlet. This advantageously means that the first primary fluid flow is established upstream of the first amplifier inlet, so that the first amplifier inlet introduces the first fluid flow directly into the first reduced-pressure zone, and the first fluid flow does not interfere with the establishment of the reduced-pressure zone by the primary flow.

When the first amplifier chamber is a non-cyclonic chamber configured to generate the first reduced-pressure zone as a result of the venturi effect, the amplifier chamber preferably has a conical shape which narrows towards the first outlet. As the first primary flow travels downstream, the conical chamber shape serves to accelerate the axial velocity of the first primary fluid flow. By positioning the first amplifier inlet downstream of the first primary inlet, the first fluid flow from the amplifier inlet is advantageously introduced into the first amplifier chamber at a point surrounded by the accelerated first primary fluid flow, such that the venturi effect at the first amplifier inlet is increased.

When the first amplifier chamber is a cyclonic chamber configured to generate a swirling first primary fluid flow, the conical chamber shape serves to accelerate the axial velocity of the first primary fluid flow, and conservation of angular momentum also causes the swirl number of the primary fluid flow to increase towards the first outlet. The venturi effect and the low-pressure cyclone core contribute to generate the greatest negative pressure in the first reduced-pressure zone downstream of the first primary inlets. By positioning the first amplifier inlet downstream of the first primary inlet, the first fluid flow from the amplifier inlet is advantageously introduced into the first amplifier chamber at a point which experiences a higher-magnitude negative pressure than is generated further upstream.

In a particularly preferred embodiment, the first amplifier inlet is positioned in the narrow end of the frusto-conical first amplifier chamber and oriented towards the first outlet. The first amplifier inlet is preferably positioned on the central axis of the first amplifier chamber closer to the first outlet (the narrowest portion of the amplifier chamber) than the first primary inlet. The first amplifier chamber may have a length defined by the distance along the central axis between the downstream edge of the first primary inlet and the first outlet at the narrowest portion of the chamber, and the first amplifier inlet may preferably be positioned in the downstream 50% of the first amplifier chamber, preferably in the downstream-most 40%, or 30%, or 20%, or 10% of the first amplifier chamber.

The narrowed portion of the chamber which forms the first outlet may advantageously act as a constriction which creates a reduced pressure due to the venturi effect. The position of the amplifier inlet relative to the narrowest portion of the chamber (the first outlet) thus determines the contribution of the venturi effect to the overall negative pressure occurring at the first amplifier inlet.

In inhalers incorporating the present invention, the medicament is delivered into the first reduced-pressure zone entrained in the first fluid flow, through the first amplifier inlet at a downstream position which is preferably nearer the first outlet than the first primary inlet. This is contrary to the aims of DPIs in the prior art, as prior art inhalers typically aim to inject powdered medicament formulations into primary airflows upstream of a long flow pathway, so that the powder is entrained in the fast airflow and deagglomerated by crashing into the chamber walls as the primary flow passes through the device. Thus if the primary fluid flow were intended to deagglomerate the medicament, it would be desirable to inject the first fluid flow and entrained medicament as far upstream as possible, so that the medicament would benefit from the accelerating primary flow and increase in swirl as the primary flow moves downstream towards the first outlet. In the present case, however, the first fluid flow with the entrained powder is preferably introduced into the reduced pressure zone at a position in the downstream half of the chamber. This ensures that the medicament is introduced into the first reduced-pressure zone in the position of lowe first amplifier chamber does not function as a deagglomeration chamber for deagglomerating powdered medicament entrained in the first fluid flow.

The first amplifier inlet is preferably formed by an aperture in the tip of a frusto-conical inlet passage. The frusto-conical inlet passage preferably extends into the inlet end of the first amplifier chamber such that the frusto-conical inlet passage is nested within the first amplifier chamber. The first amplifier inlet is preferably positioned on the central axis of the first amplifier chamber, in the first reduced-pressure zone.

The diameter of the first amplifier inlet is preferably less than 30%, or less than 20%, or less than 10% of the major diameter of the first amplifier chamber, which is the largest diameter immediately downstream of the primary flow inlets—i.e. the diameter of the first amplifier chamber measured at the downstream-edge of the primary flow inlet.

The first amplifier inlet is preferably narrower than the first outlet. In certain embodiments, the cross-sectional area (the flow area through which the fluid flow passes) of the first outlet is preferably greater than the cross-sectional area of the first amplifier inlet, preferably at least 2× greater, or 3× greater, preferably at least 5× greater.

The ratio of the cross-sectional area of the first outlet and the cross-sectional area of the first primary inlet(s) is preferably between 0.7 and 1.4, particularly preferably between 0.7 and 1.0. This ratio of flow areas may advantageously result in both a high peak acceleration of the primary fluid flow, and an advantageously high pressure drop between the outlet and the first reduced-pressure zone.

The cross-sectional area (the flow area through which the first primary fluid flow passes) of the first primary inlet is preferably greater than the cross-sectional area of the first amplifier inlet, preferably at least 5× greater, preferably at least 10× greater. This difference in flow area ensures that the primary fluid flow has a greater volumetric flow rate than the first fluid flow.

In a particularly preferred embodiment, the first amplifier inlet is a powder inlet for directing an entrained-powder-containing first fluid flow into the first amplifier chamber. The apparatus may comprise a deagglomeration engine, or a deagglomeration chamber, upstream from, and in fluid communication with, the powder inlet. The deagglomeration engine may be configured to deagglomerate a powder formulation passing therethrough while entrained in a fluid flow, preferably entrained in an airflow.

### Cyclonic First Amplifier Chamber

The first amplifier chamber may be configured to create the first reduced-pressure zone by forming the first primary fluid flow as a cyclonic flow from the first primary inlet to the first outlet, so that the first reduced-pressure zone is formed by a low-pressure core at the centre of the cyclonic flow.

The principle of swirling (cyclonic) flow may be used to effectively amplify negative pressure by trading a reduction in flowrate. The simplest definition of a cyclone is a fluid rotating around a low pressure core. In a cyclone chamber (i.e. not necessarily only reverse-flow cyclones—this is equally applicable to uniflow/through-flow cyclones, in which the air flows into one end and exits at the other), the core pressure can be considerably lower than the driving pressure. For example, it is quite reasonable to achieve a core pressure that is 1.6× that of the driving pressure—i.e. in an inhaler if the patient inhales through a swirl chamber at a (mouth) pressure drop of −4 kPa, then the pressure in the core of the swirling vortex could in a preferred embodiment be 1.6×−4=−6.4 kPa. Higher amplifications of negative core pressure are achievable with the present invention.

In a preferred embodiment of the present invention, the first amplifier chamber is a first cyclone chamber, and the first cyclone chamber is operable to establish a cyclonic, or swirling, primary fluid flow between the first primary inlet and the first outlet in response to fluid being drawn from the first outlet.

The first amplifier chamber may be a uniflow, or through-flow, frusto-conical swirl chamber. Uniflow or “through-flow” means that the flow enters one end and exits the other-unlike a reverse flow cyclone, where the inlet and outlet are at the same end.

In a uniflow frusto-conical swirl chamber, one or more tangential inlets create a swirling flow within the chamber, and due to conservation of angular momentum, the tangential velocity of the swirling airflow increases as the effective chamber diameter reduces, due to the conical nature of the geometry. This is the basis of all conical cyclones—e.g. as used in bagless vacuum cleaners—the high centripetal acceleration created within the cyclone is able to separate small dust particles by overcoming the aerodynamic drag force exerted on them—a cyclonic separator. In a uniflow design, however, all the particles are emitted rather than collected.

The cyclonic, or swirling, nature of the first primary flow may be characterised by its “swirl number”. The swirl number S of a fluid flow is defined as the ratio of the axial flux of angular momentum to the axial flux of the axial momentum.

$$S = \frac{\int rw \vec{v} \cdot d \vec{A}}{\overline{R} \int u \vec{v} \cdot d \vec{A}} \qquad ( Eq . 1 )$$

Where the first amplifier chamber is a cyclone chamber, preferably the first primary fluid flow has a swirl number greater than 0.5, or greater than 0.6, or greater than 0.7, measured at the first outlet. Preferably the first primary fluid flow has a peak (maximum) swirl number of greater than 0.5, or greater than 0.6, or greater than 0.7, in the first amplifier chamber. A swirl number greater than 0.5 indicates that the first primary fluid flow is strongly swirling.

The first primary inlet may preferably comprise one or more tangential (tangential when viewed along axis of frusto-conical chamber) inlets spaced around a perimeter of the first amplifier chamber, the tangential inlets being configured to direct incoming primary fluid flow tangentially around the wall of the amplifier chamber to create a swirling flow within the chamber. The first primary inlet may comprise 2, 3, or 4, or 5 or more tangential inlets spaced around a perimeter of the first amplifier chamber. The or each first primary inlet may be a scroll inlet, helical inlet, slot inlet, or a combination. Scroll or slot inlets may be advantageously simple to manufacture.

The potential advantages of only having a single stage cyclonic amplifier are that; i) less flowrate is traded therefore higher flowrate is available to power the deagglomeration engine, which may be particularly advantageous for high dose masses and, ii) the manufacturing and fabrication of a single stage amplifier is likely to be simpler and cheaper than a more complex multi-stage amplifier.

As described below, further stages can be added to continue to amplify the driving pressure, using the same principle-albeit the flowrate through each additional stage must reduce each time another is added. Allowing too much additional axial flow (i.e. through the amplifier inlet and not through the tangential primary inlets) will prevent the optimum development of the swirl, and reduce the overall pressure amplification.

### Non-Cyclonic First Amplifier Chamber

Instead of being a cyclonic chamber, the first amplifier chamber may alternatively be configured to create the first reduced-pressure zone as a result of the venturi effect, by constricting the cross-section of the first amplifier chamber between the first primary inlet and the first outlet.

The first primary inlet and the first amplifier chamber are configured to channel the first primary fluid flow between the first primary inlet and the first outlet. The first primary inlet and the first amplifier chamber may be configured to establish the first primary fluid flow as a sheath flow between the first primary inlet and the first outlet.

The first primary inlet and the first amplifier chamber may be configured so that the axial component of the first primary fluid flow is greater than the tangential component of the first primary fluid flow. The swirl number S of the first primary fluid flow, defined as the ratio of the axial flux of angular momentum to the axial flux of the axial momentum, may be less than 0.5, or less than 0.4, or less than 0.3, or less than 0.2, measured at the first outlet. Preferably the first primary fluid flow has a peak (maximum) swirl number of less than 0.5, or less than 0.4, or less than 0.3, or less than 0.2, in the first amplifier chamber. The swirl number S being less than 0.5 indicates that the swirl in the first primary flow is weak to moderate, unlike the strongly swirling flow that would be established by a cyclonic amplifier chamber.

The first primary inlet may be configured to direct fluid into the first amplifier chamber in a radial or axial direction. For example the first primary inlet may surround the first amplifier inlet, or a plurality of first primary inlets may be arranged around the first amplifier inlet.

### Second Amplifier Chamber

A particular advantage of the present invention is that the negative pressure may be amplified in a stepwise fashion by adding one or more additional amplifier chambers in series with the first amplifier chamber. Additional amplifier chambers are preferably added upstream of the first amplifier chamber, so in the following description, the first amplifier chamber is always the most-downstream amplifier chamber in the apparatus.

When a second amplifier chamber is added in series with the first amplifier chamber, the second amplifier is preferably arranged relative to the first amplifier so that, in use, the first reduced-pressure zone in the first amplifier chamber drives a second primary fluid flow in the second amplifier chamber, which in turn establishes a second reduced-pressure zone having a larger negative pressure than the first reduced-pressure zone. In this manner, the negative pressure applied at the apparatus outlet is amplified a first time by the first amplifier to form a greater negative pressure at the first reduced-pressure zone, and the second amplifier then amplifies that negative pressure further to form a second reduced-pressure zone.

The apparatus may additionally comprise a second amplifier chamber upstream of the first amplifier chamber, the second amplifier chamber comprising a second amplifier inlet, a second primary inlet, and a second outlet which forms, or is in fluid connection with, the first amplifier inlet of the first amplifier chamber.

The structure and function of the second amplifier chamber may be substantially as described above in relation to the first amplifier chamber.

The negative pressure amplification apparatus is preferably configured to generate a pressure drop between the first outlet and the amplifier inlet which is the furthest upstream (which is the second amplifier inlet in a two-stage apparatus), such that in response to fluid being drawn from the apparatus outlet by an outlet negative pressure, the amplifier inlet of the furthest-upstream amplifier chamber experiences an amplifier inlet negative pressure that is greater in magnitude than the outlet negative pressure.

The first amplifier chamber is preferably configured to generate a pressure drop between the first outlet and the first amplifier inlet, such that in response to fluid being drawn from the apparatus outlet by an outlet negative pressure, the first reduced-pressure zone of fluid at the first amplifier inlet experiences a first amplifier inlet negative pressure that is greater in magnitude than the outlet negative pressure. The second amplifier chamber is preferably configured to generate a further pressure drop between the second outlet (which forms the first amplifier inlet) and the second amplifier inlet, such that in response to fluid being drawn from the apparatus outlet by the outlet negative pressure, the second reduced-pressure zone of fluid at the second amplifier inlet experiences a second amplifier inlet negative pressure that is greater in magnitude than the first amplifier inlet negative pressure and the outlet negative pressure.

The second amplifier chamber is preferably configured to establish, in response to fluid being drawn from the apparatus outlet, a second primary fluid flow from the second primary inlet through the second amplifier chamber and through the second outlet into the first amplifier chamber, and to create a second reduced-pressure zone of fluid at the second amplifier inlet. The second reduced-pressure zone may then draw a second fluid flow into the apparatus through the second amplifier inlet. In preferred embodiments, powdered medicament may be entrained in the second fluid flow which is drawn through the second amplifier inlet.

Similarly to the first amplifier chamber, the second amplifier chamber may be configured to create the first reduced-pressure zone by forming the second primary fluid flow as a cyclonic flow from the second primary inlet to the second outlet, so that the second reduced-pressure zone is formed by a low-pressure core at the centre of the cyclonic flow. Alternatively the second amplifier chamber may be configured to create the second reduced-pressure zone as a result of the venturi effect, by constricting the cross-section of the first amplifier chamber between the first primary inlet and the first outlet. These embodiments are discussed further below.

The second amplifier chamber is preferably configured to generate a pressure drop between the second outlet and the second amplifier inlet, such that in response to fluid being drawn from the second outlet, the second reduced-pressure zone of fluid at the second amplifier inlet experiences a second inlet negative pressure that is greater in magnitude than the negative pressure at the first reduced-pressure zone.

The second amplifier chamber comprises a conduit having an inlet end and an outlet end, the amplifier chamber being tapered from a larger diameter at the inlet end to a smaller diameter at the outlet end. The second outlet is positioned at the outlet end. The second amplifier chamber may be open at the inlet end, so that the inlet end of the conduit forms the second primary inlet. Alternatively, the second amplifier chamber may comprise a back wall closing the inlet end, and the second primary inlet may be formed in the back wall or in a side wall of the chamber.

The second amplifier inlet is preferably positioned downstream of the second primary inlet, such that fluid entering the second amplifier chamber through the second amplifier inlet is introduced to the second amplifier chamber downstream of the position at which the second primary fluid flow enters the second amplifier chamber through the second primary inlet.

The second amplifier inlet is preferably formed by an aperture in the tip of a frusto-conical inlet passage. The second amplifier inlet is preferably positioned on the central axis of the second chamber, in the second reduced-pressure zone.

Preferably, the second amplifier chamber is frusto-conical, with the second outlet formed by an aperture at the narrow end of the frusto-conical second chamber.

The second amplifier inlet is preferably configured so that the second fluid flow is a non-swirling fluid flow with a swirl number of less than 0.2, or less than 0.1, at the second amplifier inlet.

Particularly preferably, the first and second amplifier chambers are both frusto-conical and aligned coaxially with one another along a central axis. The outlet end of the second frusto-conical amplifier chamber may preferably be nested within the inlet end of the first frusto-conical chamber.

The second outlet is preferably formed by the narrowest portion of the frusto-conical second amplifier chamber. In a preferred embodiment, the second amplifier inlet may be positioned in or near the narrowest part of the frusto-conical second amplifier chamber. For example the second amplifier inlet may be positioned on the central axis of the second amplifier chamber closer to the second outlet than the second primary inlet. The second amplifier chamber preferably has a length defined by the distance along the central axis between the downstream-edge of the second primary inlet, and the second outlet at the narrow downstream end of the chamber, and the second amplifier inlet may preferably be positioned in the downstream 50% of the second amplifier chamber, preferably in the downstream-most 40%, or 30%, or 20%, or 10% of the second amplifier chamber.

The second amplifier inlet is preferably positioned in the second amplifier chamber, upstream of, or level with, the second outlet. In some embodiments, however, the second amplifier inlet may be positioned downstream of the second outlet, meaning that the second amplifier inlet is downstream of the first amplifier inlet through which the second primary flow enters the first amplifier chamber from the second amplifier chamber.

The diameter of the second amplifier inlet is preferably less than 30%, or less than 20%, or less than 10% of the major diameter of the second amplifier chamber, which is the largest diameter immediately downstream of the primary flow inlets—i.e. the diameter of the second amplifier chamber measured at the downstream-edge of the primary flow inlet.

The second amplifier inlet is preferably narrower than the second outlet (which also acts as the first amplifier inlet). In certain embodiments, the cross-sectional area (the flow area through which the fluid flow passes) of the second outlet is preferably greater than the cross-sectional area of the second amplifier inlet, preferably at least 2× greater, or 3× greater, preferably at least 5× greater.

The ratio of the cross-sectional area of the second outlet and the cross-sectional area of the second primary inlet(s) is preferably between 0.7 and 1.4, particularly preferably between 0.7 and 1.0. This ratio of flow areas may advantageously result in both a high peak acceleration of the primary fluid flow, and an advantageously high pressure drop between the outlet and the first reduced-pressure zone.

The cross-sectional area (the flow area through which the second primary fluid flow passes) of the second primary inlet is preferably greater than the cross-sectional area of the second amplifier inlet, preferably at least 5× greater, preferably at least 10× greater. This difference in flow area ensures that the second primary fluid flow has a greater volumetric flow rate than the second fluid flow.

Although described here with a second amplifier chamber, the apparatus may optionally comprise further amplifier chambers arranged in series upstream of the first and second amplifier chambers. For example the apparatus may comprise a third amplifier chamber arranged upstream of the second amplifier chamber, with the outlet of the third amplifier chamber acting forming the second amplifier inlet. The third, and any additional, amplifier chambers may have the same features described above in relation to the first and/or second amplifier chambers.

Each amplifier chamber in the series preferably progressively amplifies, at its reduced-pressure zone, a negative pressure applied, in use, to outlet of that amplifier, so that a negative pressure applied at the apparatus outlet is progressively amplified by each amplifier in the series.

Preferably each amplifier chamber in the apparatus is unidirectional.

In a particularly preferred embodiment, the amplifier inlet of the most-upstream amplifier chamber is a powder inlet for directing an entrained-powder-containing fluid flow into the most-upstream amplifier chamber. The apparatus may comprise a deagglomeration engine, or a deagglomeration chamber, upstream from, and in fluid communication with, the powder inlet. The deagglomeration engine is preferably configured to deagglomerate a powder formulation passing therethrough while entrained in a fluid flow, preferably entrained in an airflow.

### Cyclonic Second Amplifier Chamber

The second amplifier chamber may be configured to create the second reduced-pressure zone by forming the second primary fluid flow as a cyclonic flow from the second primary inlet to the second outlet, so that the second reduced-pressure zone is formed by a low-pressure core at the centre of the cyclonic flow.

The principle of swirling (cyclonic) flow may be used to effectively amplify negative pressure by trading a reduction in flowrate. The simplest definition of a cyclone is a fluid rotating around a low pressure core. In a cyclone chamber (i.e. not necessarily only reverse-flow cyclones—this is equally applicable to uniflow/through-flow cyclones, in which the air flows into one end and exits at the other), the core pressure can be considerably lower than the driving pressure. For example, it is quite reasonable to achieve a core pressure that is 1.6× that of the driving pressure—i.e. in an inhaler if the patient inhales through a swirl chamber at a (mouth) pressure drop of −4 kPa, then the pressure in the core of the swirling vortex could in a preferred embodiment be 1.6×−4=−6.4 kPa. Higher amplifications of negative core pressure are achievable with the present invention.

In a preferred embodiment of the present invention, the second amplifier chamber is a second cyclone chamber, and the second cyclone chamber is operable to establish a cyclonic, or swirling, primary fluid flow between the second primary inlet and the second outlet in response to fluid being drawn from the second outlet.

The second amplifier chamber may be a uniflow, or through-flow, frusto-conical swirl chamber.

Where the second amplifier chamber is a cyclone chamber, preferably the second amplifier chamber is configured so that the second primary fluid flow has a swirl number greater than 0.5, or 0.6, or 0.7, measured at the second outlet. Preferably the second primary fluid flow has a peak (maximum) swirl number of greater than 0.5, or 0.6, or 0.7, in the second amplifier chamber. A swirl number greater than 0.5 indicates that the second primary fluid flow is strongly swirling. The swirl number of the primary flow may be varied by varying the relative dimensions of the cyclone chamber, in particular the major diameter and the narrower outlet diameter of the chamber.

The second primary inlet may preferably comprise one or more tangential (tangential when viewed along axis of frusto-conical chamber) inlets spaced around a perimeter of the second amplifier chamber, the tangential inlets being configured to direct incoming primary fluid flow tangentially around the wall of the amplifier chamber to create a swirling flow within the chamber. The second primary inlet may comprise 2, 3, or 4, or 5 or more tangential inlets spaced around a perimeter of the second amplifier chamber. The or each second primary inlet may be a scroll inlet, helical inlet, slot inlet, or a combination. Scroll or slot inlets may be advantageously simple to manufacture.

In embodiments comprising more than one amplifier, preferably at least one of the amplifiers in the device is a cyclonic amplifier which is operable to establish a cyclonic primary fluid flow through that amplifier chamber in response to fluid being drawn from the apparatus outlet, preferably in which the swirl number S of the primary fluid flow through that amplifier is greater than 0.5.

Preferably at least one of the first amplifier chamber and the second amplifier chamber is a cyclone chamber operable to establish a cyclonic primary fluid flow through that amplifier chamber in response to fluid being drawn from the apparatus outlet, preferably in which the swirl number S of the primary fluid flow through that amplifier is greater than 0.5. For example the downstream amplifier (the first amplifier) may be a cyclonic amplifier, while the upstream amplifier (the second amplifier) is a non-cyclonic amplifier. Alternatively the downstream amplifier (the first amplifier) may be a non-cyclonic amplifier, while the upstream amplifier (the second amplifier) is a cyclonic amplifier. Alternatively the device may comprise multiple cyclonic amplifiers—for example both the first and second amplifiers may be cyclonic amplifiers.

Preferably at least one of the first amplifier chamber and the second amplifier chamber is a uniflow frusto-conical swirl chamber, in which the primary inlet to that amplifier chamber comprises one or more tangential inlets which are configured to create a swirling flow within that amplifier chamber.

### Non-Cyclonic Second Amplifier Chamber

In a particularly preferred embodiment, the apparatus comprises a second amplifier chamber which is configured to create the second reduced-pressure zone as a result of the venturi effect, by constricting the cross-section of the second amplifier chamber between the second primary inlet and the second outlet.

The second primary inlet and the second amplifier chamber are configured to channel the second primary fluid flow between the second primary inlet and the second outlet.

Particularly preferably, the second primary inlet and the second amplifier chamber are configured to establish the second primary fluid flow as a sheath flow between the second primary inlet and the second outlet.

The second primary inlet and the second amplifier chamber may be configured so that the axial component of the second primary fluid flow is greater than the tangential component of the second primary fluid flow. The swirl number S of the second primary fluid flow, defined as the ratio of the axial flux of angular momentum to the axial flux of the axial momentum, is less than 0.5, or less than 0.4, or less than 0.3, or less than 0.2, measured at the second outlet. Preferably the second primary fluid flow has a peak (maximum) swirl number of less than 0.5, or less than 0.4, or less than 0.3, or less than 0.2, in the second amplifier chamber. The swirl number S being less than 0.5 indicates that the swirl in the second primary flow is weak to moderate, unlike the strongly swirling flow that would be established by a cyclonic amplifier chamber.

The second amplifier chamber may optionally comprise one or more vanes configured to direct the second primary fluid flow towards the second outlet. The one or more vanes may optionally be aligned parallel with the central axis of the second amplifier chamber, which may advantageously help to stabilise and direct the sheath flow through the second amplifier chamber. The one or more vanes may alternatively be arranged helically around the second amplifier chamber. The one or more vanes may be configured to have an upstream end which is parallel to the axis of the apparatus, and to have a downstream end which is angled relative to the axis. The angled downstream end may be angled at 25 degrees or less relative to the central axis of the second amplifier chamber, or 20 degrees or less, or 15 degrees or less, or 10 degrees or less relative to the central axis of the second amplifier chamber. Particularly preferably the one or more vanes may be angled at between 2.5 and 7.5 degrees relative to the central axis of the second amplifier chamber. By positioning one or more angled vanes in the second amplifier chamber, a slight swirl may be imparted to the second primary fluid flow passing through the second amplifier chamber, without establishing a highly-swirling cyclonic flow. The vanes may provide some flow resistance to restrict the flow rate of the second primary flow. In some embodiments, the presence of vanes in the second amplifier chamber may prevent the mixing flows from becoming unstable. The second primary flow is preferably reasonably isokinetic to the first primary flow and the second fluid flow.

In a particularly preferred embodiment, the second primary fluid flow has a swirl number which falls between the swirl number of the second fluid flow (in which a medicament is entrained in preferred embodiments) and the swirl number of the first primary flow. In a particularly preferred embodiment for example, the first primary flow is a cyclonic flow with a swirl number greater than 0.5 at the first outlet, the second fluid flow is a non-swirling fluid flow with a swirl number of less than 0.2, or 0.1 at the second amplifier inlet, and the second primary flow is a sheath flow which has a swirl number (measured at the second outlet) which is greater than that of the second fluid flow, but less than that of the first primary flow.

The second primary inlet may be configured to direct fluid into the second amplifier chamber in a radial or axial direction, and to direct the fluid flow along the central axis of the chamber towards the second outlet. For example the second primary inlet may surround the second amplifier inlet, or a plurality of second primary inlets may be arranged around the second amplifier inlet.

In embodiments comprising more than one amplifier, preferably at least one of the amplifiers in the device is a non-cyclonic amplifier which is configured so that the axial component of the primary fluid flow is greater than the tangential component of the primary fluid flow through that amplifier chamber in response to fluid being drawn from the apparatus outlet. The primary inlet and the amplifier chamber of at least one amplifier may be configured so that the axial component of the primary fluid flow is greater than the tangential component of the primary fluid flow through that amplifier. The swirl number S of the primary fluid flow through that amplifier, defined as the ratio of the axial flux of angular momentum to the axial flux of the axial momentum, is preferably less than 0.5, or less than 0.4, or less than 0.3, or less than 0.2, measured at the second outlet. Preferably the primary fluid flow has a peak (maximum) swirl number of less than 0.5, or less than 0.4, or less than 0.3, or less than 0.2, in that amplifier chamber.

Particularly preferably, at least one of the amplifiers in the device is a non-cyclonic amplifier which is configured so that the primary fluid flow through that amplifier is a sheath flow between the primary inlet and the outlet of that amplifier.

For example the downstream amplifier (the first amplifier) may be a cyclonic amplifier, while the upstream amplifier (the second amplifier) is a non-cyclonic amplifier. Alternatively the downstream amplifier (the first amplifier) may be a non-cyclonic amplifier, while the upstream amplifier (the second amplifier) is a cyclonic amplifier. Alternatively the device may comprise multiple non-cyclonic amplifiers—for example both the first and second amplifiers may be non-cyclonic amplifiers.

Two Stage Amplifier with Sheath Flow

According to a second aspect of the invention there is provided a particularly preferred negative pressure amplification apparatus, comprising:
a first amplifier chamber comprising a first amplifier inlet, a first primary inlet, and a first outlet, in which the first outlet forms, or is in fluid connection with, an apparatus outlet; and
a second amplifier chamber upstream of the first amplifier chamber, the second amplifier chamber comprising a second amplifier inlet, a second primary inlet, and a second outlet which forms, or is in fluid connection with, the first amplifier inlet of the first amplifier chamber. The first amplifier chamber is configured to establish, in response to fluid being drawn from the apparatus outlet, a first primary fluid flow from the first primary inlet to the first outlet, and to create a first reduced-pressure zone of fluid at the first amplifier inlet. The apparatus is additionally configured to establish, in response to the first reduced-pressure zone, a second primary fluid flow from the second primary inlet to the second outlet and into the first amplifier chamber through the first amplifier inlet, wherein a second reduced-pressure zone of fluid is created at the second amplifier inlet. The second amplifier chamber is configured so that the axial component of the second primary fluid flow is greater than the tangential component of the second primary fluid flow.

The negative pressure amplification apparatus may thus correspond to the negative pressure amplifier apparatus of the first aspect, comprising a first amplifier chamber and a second amplifier chamber upstream of the first amplifier chamber, wherein the second amplifier chamber is a non-cyclonic amplifier chamber.

The second amplifier chamber is preferably configured to establish, in response to fluid being drawn from the apparatus outlet, a second primary fluid flow from the second primary inlet through the second amplifier chamber and through the second outlet into the first amplifier chamber, and to create a second reduced-pressure zone of fluid at the second amplifier inlet. The second reduced-pressure zone may in turn draw a second fluid flow into the apparatus through the second amplifier inlet. In preferred embodiments, powdered medicament may be entrained in the second fluid flow which is drawn through the second amplifier inlet.

The second amplifier chamber, and/or the second amplifier inlet, may be configured so that the axial component of the second fluid flow is greater than the tangential component of the second fluid flow.

In the second aspect, the first amplifier chamber may be any first amplifier chamber as described in relation to the first aspect. For example the first amplifier chamber may be a cyclonic amplifier chamber, or a non-cyclonic amplifier chamber.

The second amplifier chamber, however, is non-cyclonic in the second aspect of the invention. The inventors have surprisingly found that significant performance benefits can be provided by incorporating a second amplifier which generates a sheath flow as the second primary fluid flow.

Particularly preferably, the second primary inlet and the second amplifier chamber are configured to establish the second primary fluid flow as a sheath flow between the second primary inlet and the second outlet.

The second primary inlet and the second amplifier chamber may be configured so that the axial component of the second primary fluid flow is greater than the tangential component of the second primary fluid flow. The swirl number S of the second primary fluid flow, defined as the ratio of the axial flux of angular momentum to the axial flux of the axial momentum, is less than 0.5, or less than 0.4, or less than 0.3, or less than 0.2, or 0.1, measured at the second outlet. Preferably the second primary fluid flow has a peak (maximum) swirl number of less than 0.5, or less than 0.4, or less than 0.3, or less than 0.2, or less than 0.1, in the second amplifier chamber. The swirl number S being less than 0.5 indicates that the swirl in the second primary flow is weak to moderate, unlike the strongly swirling flow that would be established by a cyclonic amplifier chamber.

The second amplifier chamber may optionally comprise one or more vanes configured to direct the second primary fluid flow towards the second outlet. The one or more vanes may optionally be aligned parallel with the central axis of the second amplifier chamber, which may advantageously help to stabilise and direct the sheath flow through the second amplifier chamber. The one or more vanes may alternatively be arranged helically around the second amplifier chamber. The one or more vanes may be configured to have an upstream end which is parallel to the axis of the apparatus, and to have a downstream end which is angled relative to the axis. The angled downstream end may be angled at 25 degrees or less relative to the central axis of the second amplifier chamber, or 20 degrees or less, or 15 degrees or less, or 10 degrees or less relative to the central axis of the second amplifier chamber. Particularly preferably the one or more vanes may be angled at between 2.5 and 7.5 degrees relative to the central axis of the second amplifier chamber.

By positioning one or more angled vanes in the second amplifier chamber, a slight swirl may be imparted to the second primary fluid flow passing through the second amplifier chamber, without establishing a highly-swirling cyclonic flow. The vanes may provide some flow resistance to restrict the flow rate of the second primary flow. In some embodiments, the presence of vanes in the second amplifier chamber may prevent “tripping” of the fluid flow. The second primary inlet may be configured to direct fluid into the second amplifier chamber in a radial or axial direction, and to direct the fluid flow along the central axis of the chamber towards the second outlet. For example the second primary inlet may surround the second amplifier inlet, or a plurality of second primary inlets may be arranged around the second amplifier inlet.

Particularly preferably, the first amplifier chamber is a cyclonic amplifier chamber as described in relation to the first aspect, while the second amplifier chamber is configured to direct the second primary fluid flow as a sheath flow from the second primary inlets to the second outlet. The inventors have found that the combination of a downstream cyclonic amplifier with an upstream sheath-flow amplifier, or “venturi” amplifier, provides the best performance of the possible amplifier combinations.

The first amplifier chamber may preferably be a cyclone chamber which is configured to generate a swirling first primary flow so that the first primary fluid flow has a swirl number greater than 0.5, or greater than 0.6, or greater than 0.7, measured at the first outlet. A swirl number greater than 0.5 indicates that the first primary fluid flow is strongly swirling.

In a particularly preferred embodiment, the second primary fluid flow has a swirl number which falls between the swirl number of the second fluid flow (in which a medicament is entrained in preferred embodiments) and the swirl number of the first primary flow. In a particularly preferred embodiment for example, the first primary flow is a cyclonic flow with a swirl number greater than 0.5 at the first outlet, the second fluid flow is a non-swirling fluid flow with a swirl number of less than 0.2, or 0.1 at the second amplifier inlet, and the second primary flow is a sheath flow which has a swirl number (measured at the second outlet) which is greater than that of the second fluid flow, but less than that of the first primary flow. A strongly swirling first primary flow may induce a slight degree of swirl to the second primary flow even when the second amplifier chamber is configured to establish a non-swirling sheath flow. The strongly swirling first primary flow may cause the second primary flow to have a low swirl number between that of the highly-swirling first primary flow and the non-swirling second fluid flow entering through the second amplifier inlet.

The features of the negative pressure amplifier apparatus described in relation to the first aspect of the invention apply equally to the apparatus of the second aspect.

### Inhaler

According to a third aspect of the invention there is provided an inhaler apparatus for delivering a medicament to a patient, comprising a negative pressure amplification apparatus according to any preceding aspect of the invention. The inhaler preferably comprises, or is couplable to, a source of medicament such that in use, the medicament is entrained in a fluid flow upstream of the or each amplifier chamber and delivered into the most-upstream amplifier chamber through the amplifier inlet.

The inhaler may preferably be a dry powder inhaler (DPI) configured to entrain a dry powdered medicament, and to deliver out of the apparatus outlet an airflow containing the entrained medicament.

The inhaler is preferably a passive inhaler, which does not have its own energy source. Delivery of the powdered medicament to the patient is preferably performed using only the inspiratory energy of the patient.

The inhaler preferably comprises a mouthpiece positioned at the apparatus outlet, so that a patient can apply a negative pressure to the apparatus outlet by inhaling, or drawing, air out of the mouthpiece. The mouthpiece preferably surrounds the first outlet. The first outlet may form the outlet of the inhaler, or an outlet passage such as a reverse frusto-conical outlet passage may be positioned between the first outlet and the outlet of the inhaler.

The inhaler preferably comprises an inhaler inlet upstream of the source of medicament, the inhaler inlet being in fluid communication with the source of medicament and then the amplifier inlet of the most-upstream amplifier chamber in the inhaler. The inhaler inlet may be configured to direct a flow of air into contact with the source of medicament. The inhaler may additionally comprise one or more primary inhaler inlets configured to direct a flow of air into the primary inlets of the or each amplifier chamber.

The primary inlet of each amplifier chamber are preferably air inlets, and the medicament is configured to be entrained in an airflow and drawn into the most-upstream amplifier chamber through the most-upstream amplifier inlet.

In use, a patient inhales through the mouthpiece, which creates a negative pressure at the apparatus outlet, and draws the first primary flow of air through the apparatus, creating the first reduced-pressure zone at the first amplifier inlet. The negative pressure at the first reduced-pressure zone has a greater magnitude than the patient's inhalation, so the pressure drop across the medicament (between the inhaler inlet upstream of the medicament and the first amplifier inlet downstream of the medicament) is amplified. The first fluid flow is drawn in through the inhaler inlet, entrains the medicament and is drawn downstream through the first amplifier inlet by the amplified negative pressure in the first amplifier chamber. The first fluid flow (containing the medicament) and the first primary flow then combine and are drawn out of the apparatus outlet to be inhaled by the patient.

The negative pressure amplification apparatus thus advantageously amplifies the negative pressure generated by the patient, so that the negative pressure which draws the first fluid flow through the source of medicament has a greater magnitude than can be naturally generated by the patient's inspiratory energy

The inhaler is preferably configured so that the source of medicament is in fluid communication with the amplifier inlet of the most-upstream amplifier chamber.

Where two amplifier chambers are arranged in series, for example, the source of medicament should be arranged in fluid communication with the second amplifier inlet, as the second amplifier chamber is upstream of the first amplifier chamber.

With a two-stage amplifier, in use, a patient inhales through the mouthpiece, which creates a negative pressure at the apparatus outlet, and draws the first primary flow of air through the apparatus, creating the first reduced-pressure zone at the first amplifier inlet. The negative pressure at the first reduced-pressure zone has a greater magnitude than the patient's inhalation, and in turn the negative pressure in the first-reduced pressure zone draws air into the first amplifier chamber through the first amplifier inlet, which also forms the second outlet from the second amplifier chamber. This draws the second primary airflow into the second amplifier chamber through the second primary inlet, creating the second reduced-pressure zone at the second amplifier inlet. The negative pressure at the second reduced-pressure zone is greater in magnitude than that in the first reduced-pressure zone, so the pressure drop across the medicament (between the inhaler inlet upstream of the medicament and the second amplifier inlet downstream of the medicament) is amplified. In a two-amplifier inhaler, the second amplifier inlet forms a powder inlet in fluid communication with the source of powdered medicament, and with the inhaler inlet upstream of the medicament. The second fluid flow is drawn in through the inhaler inlet, entrains the medicament and is drawn downstream through the second amplifier inlet by the amplified negative pressure in the second amplifier chamber. The second fluid flow (containing the medicament) and the second primary flow then combine and are drawn out of the second outlet, to enter the first amplifier chamber as the first fluid flow. The first fluid flow is drawn downstream through the first amplifier chamber, combines with the first primary flow, and passes out of the apparatus outlet to be inhaled by the patient.

The apparatus may comprise a deagglomeration chamber, upstream from, and in fluid communication with, the powder inlet. The deagglomeration chamber is preferably configured to deagglomerate a powder formulation passing therethrough while entrained in an airflow. The deagglomeration chamber may be positioned downstream of the source of medicament and upstream of the amplifier inlet of the most-upstream amplifier chamber. Alternatively the deagglomeration chamber may contain the source of medicament. For example the deagglomeration engine may be a blister. Alternatively the deagglomeration engine may be a section of conduit comprising one or more baffles.

The features of the negative pressure amplifier apparatus described in relation to the first and second aspects of the invention apply equally to the inhaler of the third aspect.

### PREFERRED EMBODIMENTS OF THE INVENTION

Preferred embodiments of the invention will now be described by way of example, with reference to the accompanying drawings, in which:

FIG. 1: A graph illustrating the effect of input energy (energy applied to a powder) on aerosolisation (fine particle) efficiency of typical passive DPIs;

FIG. 2: A graph illustrating the relationship between inspiratory energy and height;

FIG. 3: A graph illustrating the effect of age upon pressure and flowrate;

FIG. 4: A graph showing children and adults' Mouth Pressure;

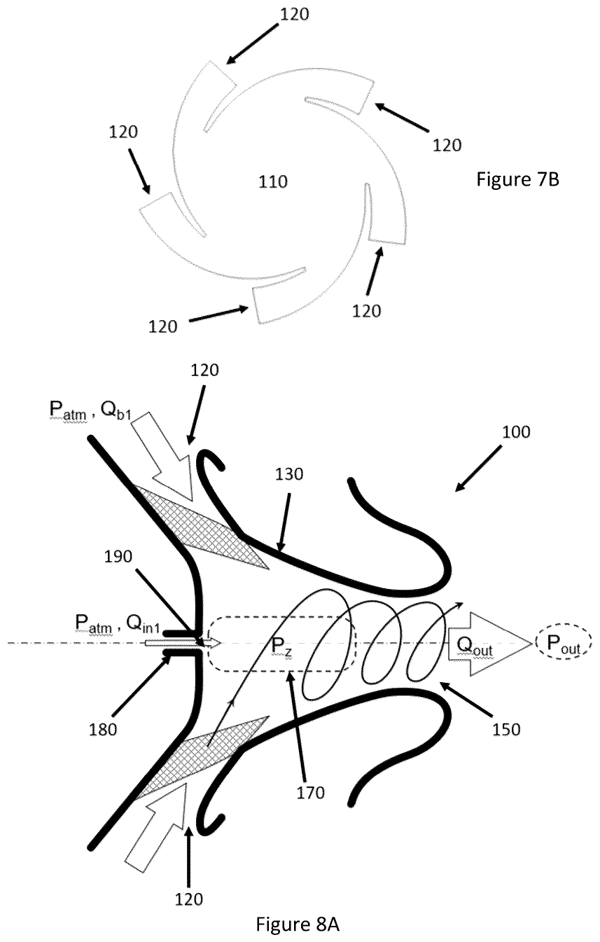

FIG. 5A: Side-on cross-section of a single-stage venturi amplifier apparatus according to a first embodiment of the present invention;

FIG. 5B is a performance chart of flow Q_{b} vs pressure P_{b} for the single-stage venturi amplifier of FIG. 5A;

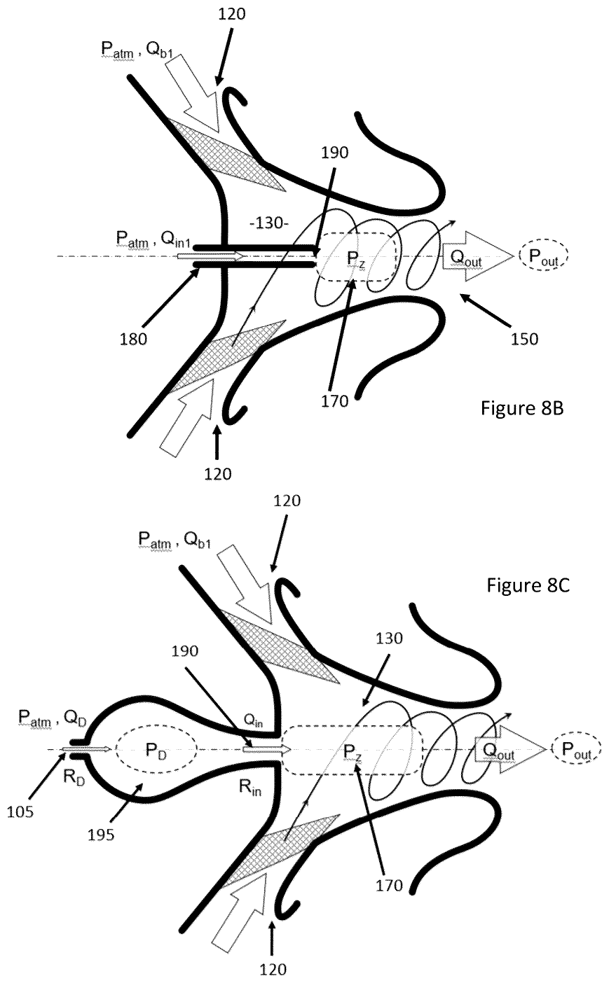

FIG. 6: Side-on cross-section of a single-stage cyclone amplifier apparatus according to the second embodiment of the present invention, with the low-pressure cyclone core illustrated by CFD;

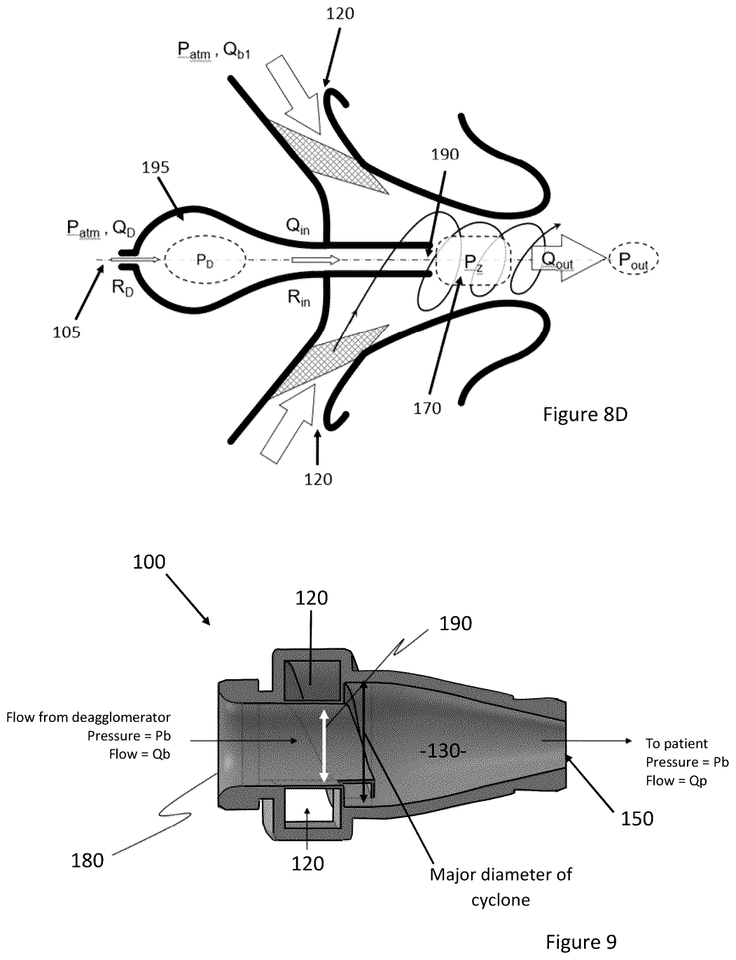

FIG. 7A: End view of a uniflow frusto-conical, twin-inlet swirl chamber usable in a second embodiment of the present invention;

FIG. 7B: End view of a uniflow frusto-conical, five-primary inlet swirl chamber usable in a second embodiment of the present invention;

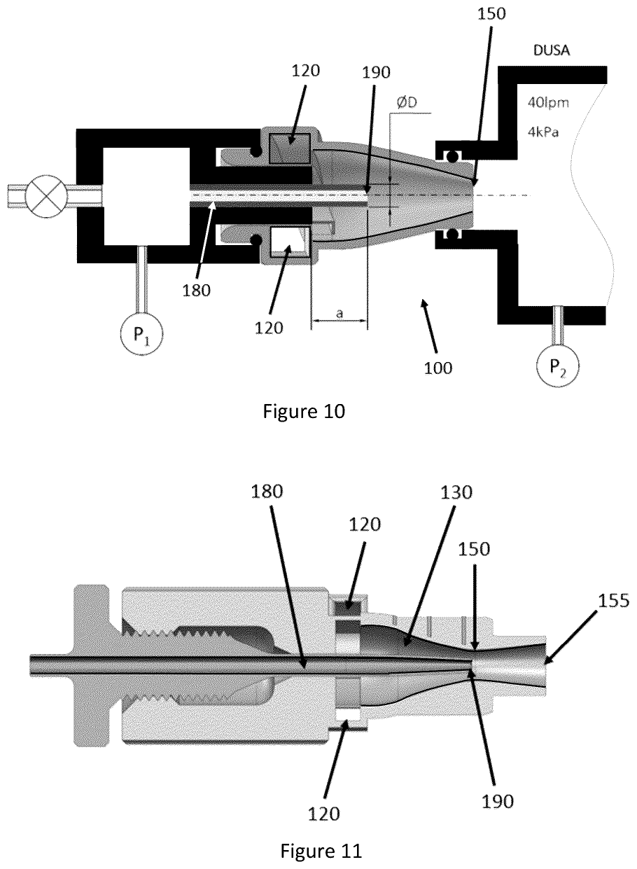

FIG. 8A: Schematic side view illustrating fluid flow through a first single-stage amplifier apparatus;

FIG. 8B: Schematic side view illustrating the single-stage amplifier apparatus of FIG. 8A, with a different amplifier inlet position;

FIG. 8C: Schematic side view illustrating the single-stage amplifier apparatus of FIG. 8A, with a deagglomerator;

FIG. 8D: Schematic side view illustrating the single-stage amplifier apparatus of FIG. 8C, with a different amplifier inlet position;

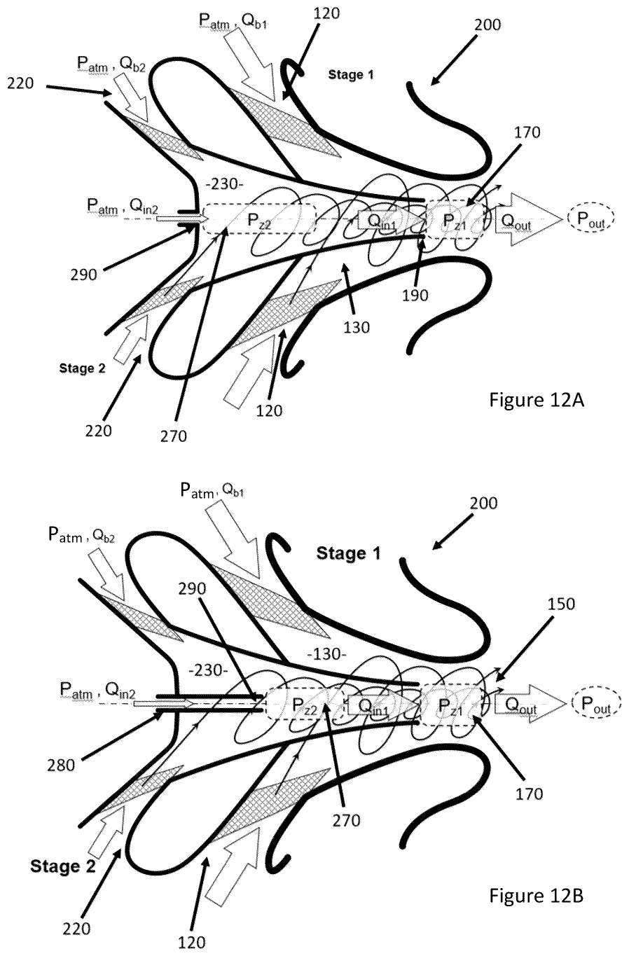

FIG. 9: Side-on cross-section of a single-stage cyclone amplifier apparatus according to an embodiment of the present invention;

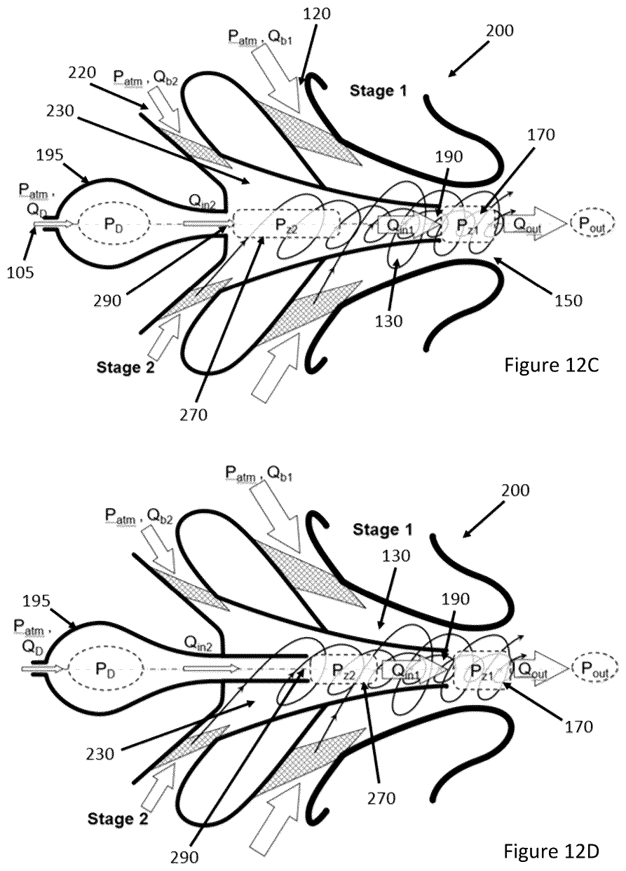

FIG. 10: Side-on cross-section of the single-stage cyclone amplifier apparatus of FIG. 9, in an experimental setup to measure fluid flow through the amplifier;

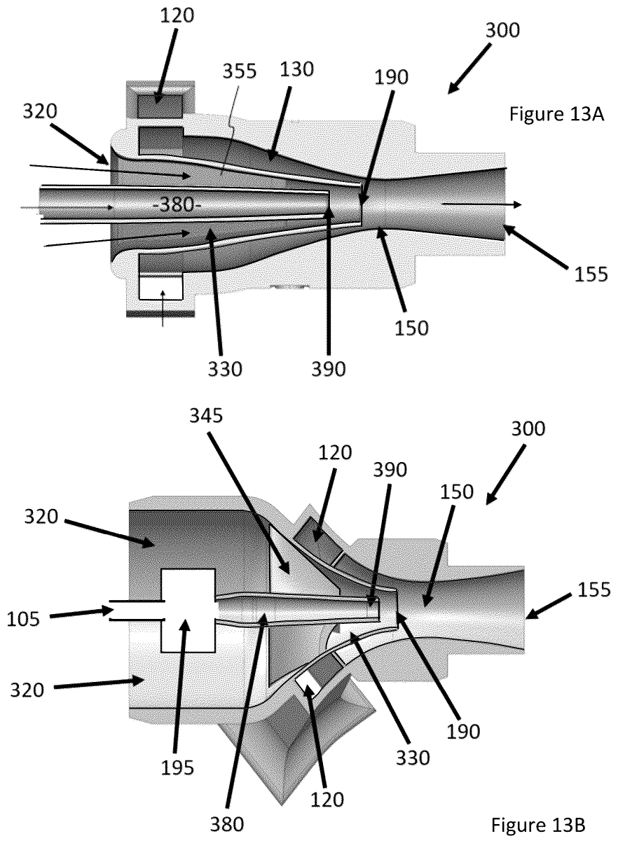

FIG. 11: Side-on cross-section of a single-stage cyclone amplifier apparatus according to a preferred embodiment of the present invention;

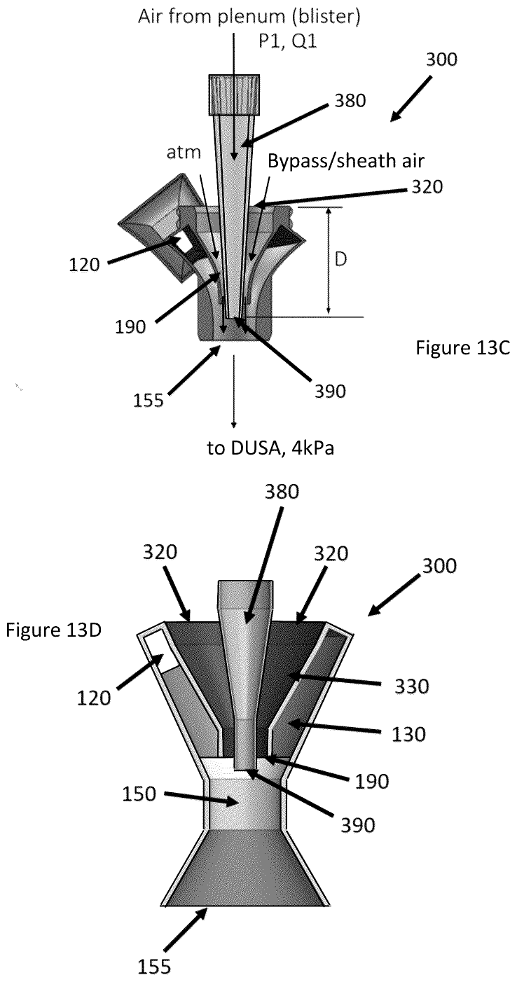

FIG. 12A: Side-on cross-section of a first two-stage cyclone amplifier apparatus according to an embodiment of the present invention;

FIG. 12B: Side-on cross-section of the two-stage cyclone amplifier apparatus of FIG. 12A, with a different second amplifier inlet position;

FIG. 12C: Side-on cross-section of the two-stage cyclone amplifier apparatus of FIG. 12A, with a deagglomerator;

FIG. 12D: Side-on cross-section of the two-stage cyclone amplifier apparatus of FIG. 12C, with a different second amplifier inlet position;

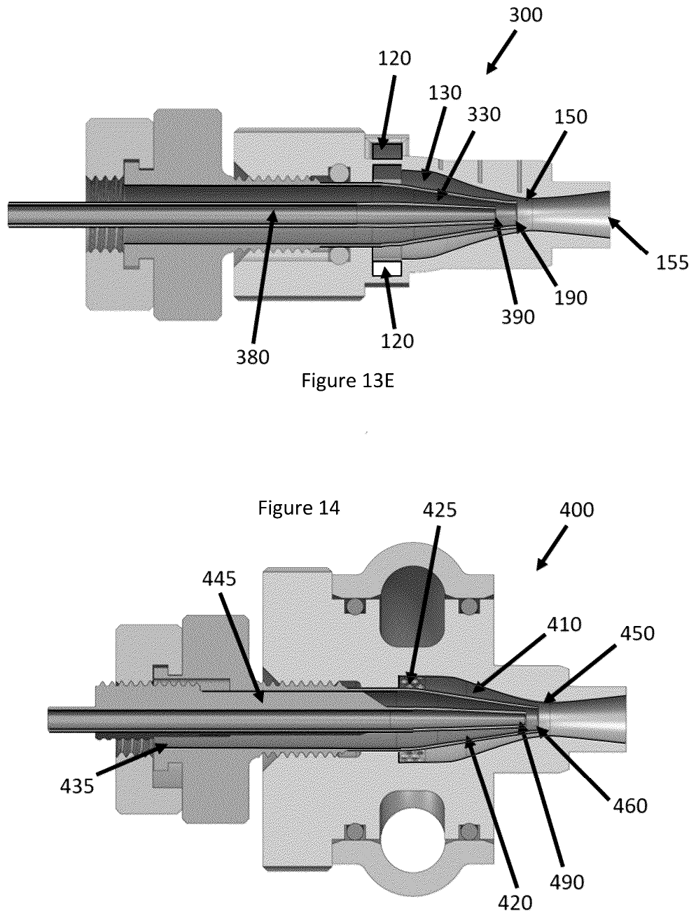

FIG. 13A: Side-on cross-section of a two-stage amplifier apparatus according to a preferred embodiment of the present invention, in which the first stage is a cyclone amplifier chamber and the second stage is a sheath flow amplifier chamber;

FIG. 13B: Side-on cross-section of a two-stage amplifier apparatus according to a preferred embodiment of the present invention, in which the first stage is a cyclone amplifier chamber and the second stage is a sheath flow amplifier chamber;

FIG. 13C: Simplified side-on cross-section of a two-stage amplifier apparatus, in which the first stage is a cyclone amplifier chamber and the second stage is a sheath flow amplifier chamber;

FIG. 13D: Side-on cross-section of a two-stage amplifier apparatus, in which the first stage is a cyclone amplifier chamber and the second stage is a sheath flow amplifier chamber;

FIG. 13E: Side-on cross section of a two-stage amplifier apparatus according to a preferred embodiment of the present invention, in which the first stage is a cyclone amplifier chamber and the second stage is a sheath flow amplifier chamber;

FIG. 13F: Schematic side-on cross-section of a two-stage amplifier apparatus according to a preferred embodiment of the present invention, in which the first stage is a cyclone amplifier chamber and the second stage is a sheath flow amplifier chamber;

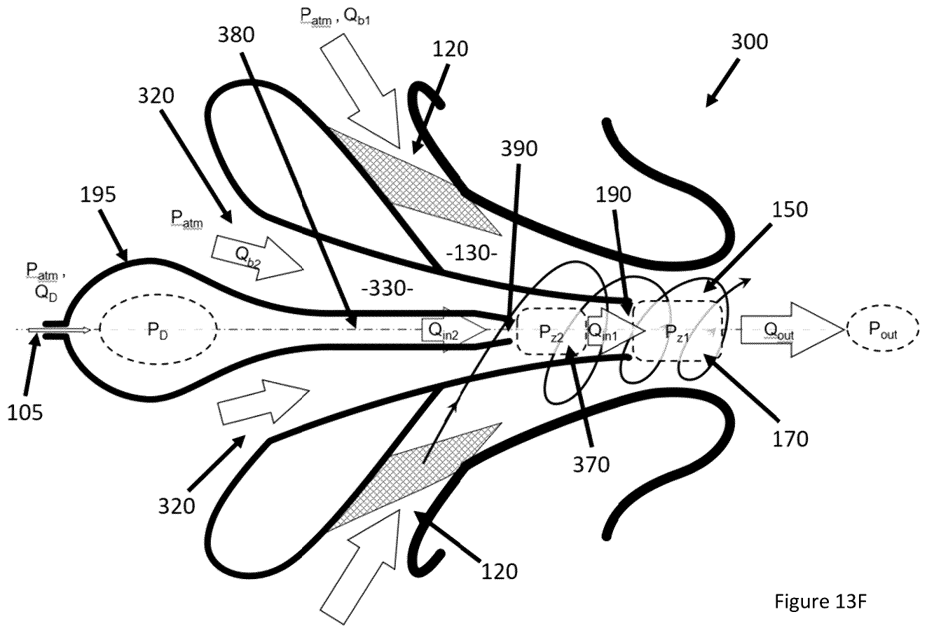

FIG. 14: Side-on cross-section of a two-stage amplifier apparatus according to an embodiment of the present invention, in which the first stage is a venturi amplifier chamber and the second stage is a sheath flow amplifier chamber;

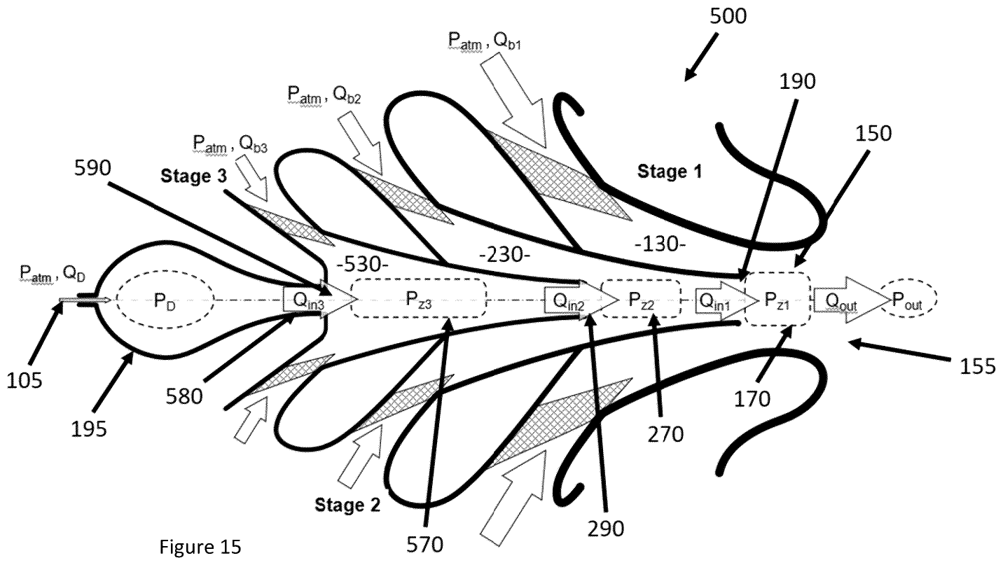

FIG. 15: Side-on cross section of a three-stage amplifier apparatus according to the present invention, in which all three amplifier chambers are cyclonic amplifier chambers;

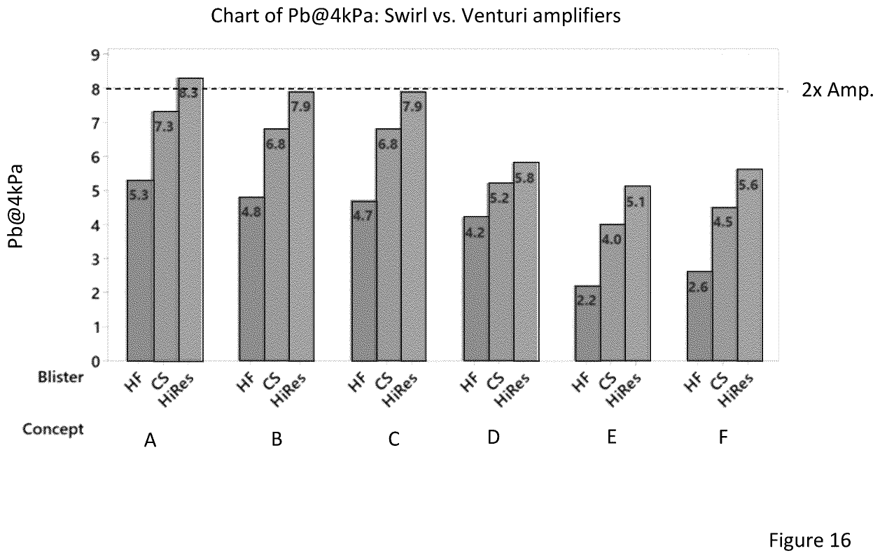

FIG. 16: Performance comparison of different amplification apparatus embodiments; and

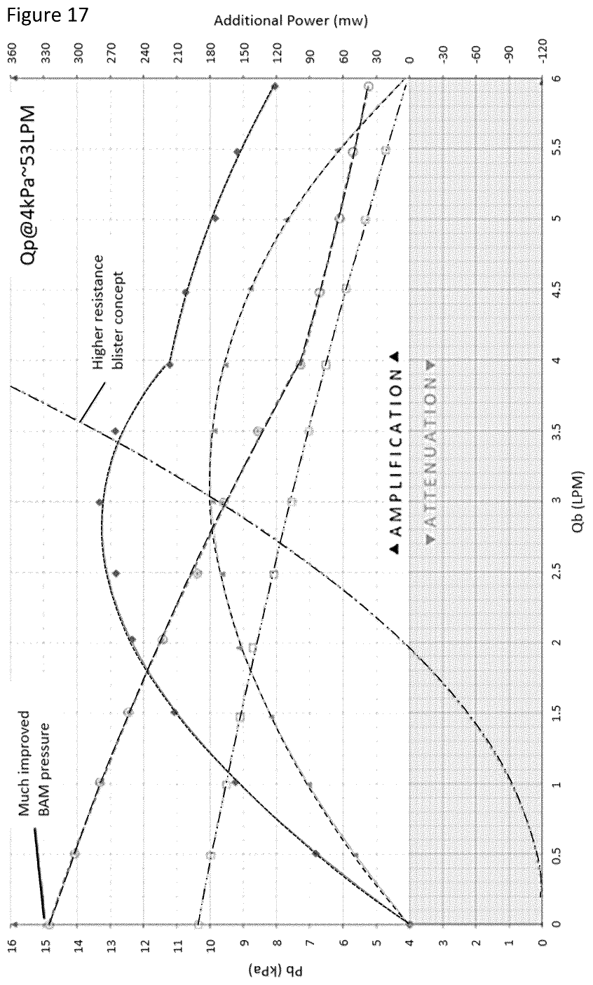

FIG. 17: Performance chart of flow Q_{b} vs pressure P_{b} and additional power for the two-stage amplifier of FIG. 13A.

### SINGLE-STAGE VENTURI AMPLIFIER

FIG. 5A illustrates a single-stage “venturi” negative pressure amplification apparatus 10 according to a first embodiment of the present invention. The apparatus 10 comprises a hollow, generally cylindrical body 20 through which a conduit extends from an inlet end 15 to an outlet end 25. At the inlet end, the hollow core of the conduit comprises a frusto-conical amplifier chamber 30, which tapers from a primary inlet 35 at the inlet end of the body 20, to a narrowed neck which forms an outlet 40 of the amplifier chamber 30. From the outlet 40, the hollow core of the conduit extends to the outlet end of the body as an outlet passage 60. An apparatus outlet 70 is formed where the outlet passage 60 reaches the outlet end of the body 20.

A hollow inlet conduit 80 is positioned on the central axis of the cylindrical body 20, and extends into the amplification chamber 30 from the inlet end of the apparatus. An upstream end of the inlet conduit is connectable in a preferred embodiment to a source of medicament. The inner diameter of the hollow inlet conduit 80 is significantly smaller than the inner diameter of the amplifier chamber, and the downstream end of the inlet conduit 80 forms an amplifier inlet 90 of the apparatus 10, which is positioned in the narrowed neck outlet 40 of the amplifier chamber.

In a preferred embodiment, the upstream end of the hollow inlet conduit 80 is coupled to a blister containing powder medicament, and/or a deagglomerator, so that the first airflow through the inlet conduit 80 is drawn through the blister and entrains the medicament.

In use, a negative pressure Pₚ (patient pressure) is applied to the apparatus outlet 70 by drawing air out of the outlet end of the body. The air drawn out of the apparatus outlet 70 has a flow rate Qₚ. This then draws a primary flow of air into the amplifier chamber 30 through the primary inlet 35. As the primary flow of air is drawn downstream, it is constricted and axially accelerated by the frusto-conical shape of the amplifier chamber, which creates a reduced-pressure zone in the narrowed neck 40 due to the venturi effect. The venturi effect creates a pressure drop between the apparatus outlet 70 and the reduced-pressure zone, such that the negative pressure applied at the apparatus outlet 70 is amplified at the reduced-pressure zone by the shape of the amplifier chamber 30.

The amplifier inlet 90 is positioned in the reduced-pressure zone, so the amplified negative pressure in the reduced-pressure zone acts to draw a first flow of air inwards through the hollow inlet conduit 80. The first flow of air thus experiences a higher-magnitude negative pressure than is being applied to the apparatus outlet 70, which means that the first flow of air is drawn into the apparatus at a higher velocity than would be possible using the negative pressure applied at the apparatus outlet 70. The first flow of air drawn through the amplifier inlet has a pressure P_{b} (blister pressure) and a flow rate Q_{b}.

Pressures discussed herein are negative pressures, although not expressed as negative numbers.

As shown in FIG. 16, this amplifier design achieved a maximum of 5.1 kPa blister pressure P_{b} in response to a patient pressure Pₚ of 4 kPa applied to the apparatus outlet, which translated to a ˜1.3× amplification of the applied negative pressure.

FIG. 5B is a performance chart of flow Q_{b} vs pressure P_{b} for the single-stage venturi amplifier of FIG. 5A, based on a patient pressure Pₚ of 4 kPa, overlaying the venturi's operating characteristics with 3 different upstream deagglomerators, each having a different flow resistance: a lower flow resistance deagglomerator (which provided the lowest flow resistance of the three); a medium flow resistance deagglomerator, and a higher flow resistance deagglomerator (which provided the highest flow resistance to the first airflow passing through the blister). The intersections in the graph of FIG. 5B are the operating points for the single-stage venturi amplifier and the coupled deagglomerator.

As shown in FIG. 5B, when the venturi amplifier 10 was coupled to the lower flow resistance deagglomerator, the P_{b} was only 2.2 kPa, so this arrangement did not amplify the negative pressure applied to the apparatus outlet. When coupled to the medium flow resistance deagglomerator, the venturi amplifier 10 produced a P_{b} of 4 kPa, equal to the input negative pressure Pₚ. When the venturi amplifier 10 was coupled to the High flow resistance deagglomerator, however, the P_{b} was 5.1 kPa, so this arrangement did amplify the negative pressure by roughly 30% relative to the pressure applied at the apparatus outlet.

### Single-Stage Cyclone Amplifier

In a second embodiment of the present invention, a cyclonic amplifier chamber is employed instead of the venturi amplifier chamber discussed above. FIGS. 6 to 11 illustrate a variety of design modifications applicable to a single-stage amplification apparatus in which the amplifier chamber is configured to generate a swirling cyclone airflow which forms a low pressure core at its centre.

All of the designs of FIGS. 6 to 11 are based on a uniflow frusto-conical amplifier chamber 130 according to a second embodiment of the present invention. In a negative pressure amplification apparatus 100, the amplifier chamber 130 comprises a frusto-conical conduit which tapers from a wide inlet end 115 to a narrowed neck which forms an outlet 150 at the narrower end of the chamber. The outlet 150 widens into an outlet passage which extends to an outlet 155 of the apparatus 100.

In order to generate a cyclonic swirling airflow, which travels in a spiral within the narrowing chamber, the amplifier chamber 130 comprises multiple tangential primary inlets 120 which are arranged to direct air tangentially into the amplifier chamber so that it travels around the sides of the chamber. As shown in FIGS. 7A and 7B, the apparatus may comprise various numbers and shapes of primary inlet 120, for example a pair of diametrically-opposed tangential inlets as shown in FIG. 7A, or five tangential primary inlets arranged around the circumference of the amplifier chamber 110. The primary inlets may be scroll inlets, helical inlets, slot inlets, or a combination of such inlets. The tangential inlets may optionally be angled relative to the axis, as shown in FIGS. 8A-8D, so that airflow entering the chamber through the primary inlets has an axial component as well as a tangential component. This creates a strongly swirling primary flow, which has a swirl number of over 0.5.

In other embodiments of cyclonic amplifier chambers, the primary inlets are tangential and arranged in a plane that is normal to the central axis of the device, i.e. the primary inlets are not angled relative to the axis as shown in FIGS. 8A to 8D. For example the primary inlets may be scroll or slot inlets, which are advantageously easier to manufacture.

As the airflow travels in a spiral down the amplifier chamber from the primary inlets, the constricting chamber walls force the airflow to accelerate both axially and tangentially, and a zone of reduced-pressure is formed along the axis at the core 175 of the cyclone, as shown in FIG. 6.

A hollow inlet conduit 180 is positioned on the central axis of the apparatus, and extends into the amplification chamber 130 from the inlet end of the apparatus. An upstream end of the inlet conduit is connectable in a preferred embodiment to a source of medicament and preferably a deagglomerator. The inner diameter of the hollow inlet conduit 180 is significantly smaller than the inner diameter of the amplifier chamber, and the downstream end of the inlet conduit 180 forms an amplifier inlet 190 of the apparatus 100.

As shown in FIG. 7A, the first amplifier inlet 190 has a smaller flow cross-section than the first outlet 150.

The position of the amplifier inlet 190 relative to the outlet can be varied, as shown in FIGS. 8A-8D. In order to ensure that the first airflow entering through the amplifier inlet 190 is being injected into the low pressure core of the cyclone and does not disrupt the formation of the low pressure zone, however, it is highly desirable that the amplifier inlet 190 is positioned downstream of the primary inlets 120. This means that the swirling primary airflow entering the amplifier chamber through the primary inlets 120 is already swirling, and has established a cyclonic flow and a low pressure core in the position where the amplifier inlet 190 injects the first airflow into the amplifier chamber.

The diameter of the amplifier inlet 190 should be small enough that it is encompassed by the diameter of the reduced-pressure zone in the amplifier chamber.

In the embodiments illustrated in FIG. 8A and FIG. 8C, the amplifier inlet 190 is positioned level with the downstream edges of the primary inlets 120. In FIGS. 8B and 8D, the inlet conduit 180 projects further downstream along the axis of the apparatus, such that the amplifier inlet 190 is positioned on the axis well downstream of the primary inlets 120. In the schematic illustration of FIGS. 8B and 8D, the amplifier inlet 190 is positioned approximately half way between the primary inlets 120 and the outlet 150, but in reality it is preferred for the amplifier inlet to be provided further along the axis towards the outlet 150.

In the embodiments of FIGS. 8A and 8B, in use, a negative pressure Pₒᵤₜ (outlet pressure) is applied to the apparatus outlet 155 and air is drawn out of the outlet at a flow rate Qₒᵤₜ. This then draws a primary flow of air into the amplifier chamber 130 through the primary inlets 120. The pressure at the primary inlets 120 is atmospheric pressure Pₐₜₘ, so the pressure drop between Pₒᵤₜ and Pₐₜₘ draws in the primary flow at a flow rate Q_{b1}. The flow through each primary inlet is [(Total Primary Flow)/n] where n is the number of inlets.

As the primary flow of air is drawn downstream, it is axially and tangentially accelerated by the constricting frusto-conical shape of the amplifier chamber. The primary airflow flows in a spiral direction around the chamber walls as it moves downstream, which creates a reduced-pressure P_{z} in a reduced-pressure zone 170 along the central axis of the apparatus, in the core of the swirling primary flow. P_{z} is lower than Pₒᵤₜ, such that there is a pressure drop between the apparatus outlet 155 and the reduced-pressure zone. The negative pressure applied at the apparatus outlet 155 is thus amplified at the reduced-pressure zone by the cyclonic primary flow and the shape of the amplifier chamber 130.

The amplifier inlet 190 is positioned in the reduced-pressure zone, so that the amplified negative pressure P_{z} in the reduced-pressure zone 170 acts to draw a first flow of air inwards through the hollow inlet conduit 180. The pressure at the upstream end of the inlet conduit 180 is atmospheric pressure Pₐₜₘ, so the pressure drop between P_{z} and Pₐₜₘ draws in the primary flow at a flow rate Qᵢₙ₁. Due to the higher-magnitude negative pressure P_{z} drawing in the first airflow, the pressure difference across the inlet conduit 180 is greater than the pressure difference between Pₒᵤₜ and Pₐₜₘ at the apparatus inlets. This means that the first flow of air is drawn into the apparatus at a higher velocity than would be possible using only the negative pressure applied at the apparatus outlet 155.

In the embodiments of FIGS. 8A and 8B, |P_{Z}|>|Pₒᵤₜ|>|Pₐₜₘ|, and P_{Z}<Pₒᵤₜ<Pₐₜₘ.

The preferred embodiments of FIGS. 8C and 8D relate to an inhaler apparatus in which the upstream end of the hollow inlet conduit 180 is shown coupled to a deagglomerator 195. An apparatus inlet 105 is provided at the upstream end of the deagglomerator 195, so that the airflow drawn through the apparatus inlet 105 is drawn through the deagglomerator before passing through the amplifier inlet 190 and reaching the amplifier chamber 130. The resistance R_{D} of the apparatus inlet 105 may differ from the resistance of the amplifier inlet 190. In particular, R_{D} is preferably larger than Rᵢₙ, so Rᵢₙ<R_{D}.

The deagglomerator may be a blister pack containing powdered medicament, so that the airflow passing through the deagglomerator entrains and deagglomerates the powder formulation.

The embodiments of FIGS. 8C and 8D operate in the same way as those shown in FIGS. 8A and 8B. In FIGS. 8C and 8D, however, the first flow of air is fed to the amplifier inlet 190 from the deagglomerator 195.

When the amplified negative pressure P_{Z} is generated in the reduced-pressure zone 170, the first airflow is drawn into the deagglomerator 195 through the apparatus inlet 105. The pressure at the apparatus inlet 105 is atmospheric pressure Pₐₜₘ, so the pressure drop between P_{z} and Pₐₜₘ draws the first flow into the deagglomerator at a flow rate Q_{D}. The negative pressure P_{D} in the deagglomerator is not as low as P_{Z} due to the pressure loss across the restriction formed by the amplifier inlet 190. Nevertheless, the pressure drop across the deagglomerator draws in the first flow of air at a higher velocity than would be possible using only the negative pressure applied at the apparatus outlet 155, and the first flow entrains and deagglomerates the powdered medicament in the deagglomerator before injecting it into the reduced-pressure zone 170. It is preferable that the resistance of the amplifier inlet 190, Rᵢₙ, is substantially lower than the apparatus inlet 190 such that the majority of the pressure loss in the system is across the apparatus inlet 190.

In the embodiments of FIGS. 8C and 8D, |P_{Z}|>|P_{D}|>|Pₒᵤₜ|>|Pₐₜₘ|, P_{Z}<P_{D}<Pₒᵤₜ<Pₐₜₘ, R_{D}>Rᵢₙ, and Q_{D}=Qᵢₙ.

The cyclonic amplifier chamber 130 is in some ways advantageous over a venturi tube approach, as it may be able to produce an increased pressure drop at the reduced-pressure zone. Rather than the primary airflow all travelling axially through the tapering amplifier chamber, in a cylonic flow the primary airflow has a component of rotation around the axis, i.e. the velocity vectors in a venturi system are parallel to the axis, while the velocity vectors in a cyclone system are not. This swirling flow arrangement may thus mean that a cyclonic amplifier chamber can “fit” a greater mass flow of air through a given space by swirling it through that space, and the resulting higher mass flowrate provides a greater energy (or power) density to draw in the first flow of air through the amplifier inlet 190. The inventors have found that the amplification performances achievable with a venturi system (in which the velocity streamlines of the primary airflow are normal to the open cross-sectional areas) can be improved by swirling the air through the same open cross-sectional areas, as swirling airflow designs can achieve a higher mass flowrate through the same given area by moving the airflow direction away from normal.

The low-pressure region is different in a cyclone amplifier chamber compared to a venturi amplifier chamber. In a venturi amplifier chamber, the low-pressure region is in the area of most constriction (the narrow neck of the amplifier chamber), while in a cyclone amplifier chamber the reduced-pressure region extends along the axis to the back wall as shown in FIG. 6. This advantageously allows for more design freedom in cyclonic designs, as the amplifier inlet 190 may be positioned in a range of positions along the central axis.

The inventors have found that, although the reduced-pressure zone at the core of the cyclonic primary flow extends along the chamber's central axis, it is advantageous to position the amplifier inlet 190 in the downstream half of the amplifier chamber 130, in or close to the narrowest portion of the tapering amplifier chamber. This may advantageously maximise the pressure drop between the outlet and the amplifier inlet 190, as the venturi effect created by the constricted outlet 150 is added to the reduced-pressure core created by the swirling primary flow. Injecting the first airflow into the amplifier chamber so far downstream is counterintuitive, as many prior art inhaler designs have typically aimed to inject medicament as far upstream as possible, and into the primary flow of air, so that the medicament is entrained in the primary flow and deagglomerated by contact with the conduit walls as it passes downstream.

FIGS. 9 and 10 show a side-on cross-section of a single-stage cyclone amplifier apparatus similar to that shown in FIGS. 8A and 8B. The experimental setup shown in FIG. 10 was used to investigate the effects of changing the diameter of the amplifier inlet 190, and varying the insertion depth of the amplifier inlet 190 along the axis of the amplifier chamber 130. In FIG. 10, P₂≡Pₒᵤₜ (outlet pressure, for example the negative pressure being drawn by a patient), P₁≡P_{b} (blister pressure, which may be termed P_{D} deagglomerator pressure—this is the negative pressure experienced in the deagglomerator, which can be used to entrain and deagglomerate a medicament).

This investigation showed that it is important to have an amplifier inlet 190 that is smaller than the major diameter of the cyclone, in order to tap into the reduced-pressure core directly. CFD investigations of the pressure profile as a function of radius (FIG. 6) showed that the pressure clearly drops off as distance away from the axis increases. In order to tap into the reduced-pressure zone, the amplifier inlet 190 is thus always positioned on the axis, and the diameter of the amplifier inlet 190 is always less than the major diameter of the cyclonic amplifier chamber. The inventors have found, however, that there is a trade-off between mass flow and core pressure—an infinitely narrow amplifier inlet is desirable in order to access the lowest pressure in the core, but with an infinitely low airflow resistance (which is of course impossible) in order to not restrict airflow from the deagglomerator 195.

FIG. 11 illustrates a side-on cross-section of another single-stage cyclone amplifier apparatus, in which the insertion depth of the amplifier inlet 190 into the amplifier chamber 130 was variable, to investigate the effects of insertion depth on negative pressure amplification. The inventors found that, surprisingly, there was a significant uplift in negative pressure amplification when the amplifier inlet 190 was positioned in the downstream half of the amplifier chamber 130, particularly when the amplifier inlet was positioned in or near the narrowest portion of the amplifier chamber 130. The reason for the increased amplification in this position may be that this is where the venturi effect caused by the constriction in the chamber contributes to the reduced-pressure cyclone core.

### Two-Stage Cyclone Amplification Apparatuses

A particular advantage of the present invention is that the negative pressure may be amplified in a stepwise fashion by adding one or more additional amplifier chambers in series with the first amplifier chamber. Additional amplifier chambers are added upstream of the first amplifier chamber, so the first amplifier chamber remains the most-downstream amplifier chamber in the apparatus.

FIGS. 12A-12D illustrate various embodiments of a two-stage cyclone amplifier apparatus 200 according to the present invention.

As shown in FIGS. 12A-12D, a frusto-conical uniflow second amplifier chamber 230 is added in series with the first amplifier chamber 130. The second amplifier chamber 230 is arranged upstream of and nested inside the first amplifier so that, in use, the first reduced-pressure zone 170 in the first amplifier chamber 130 drives a second primary fluid flow in the second amplifier chamber 230, which in turn establishes a second reduced-pressure zone 270 having a larger negative pressure P_{Z2} than P_{Z1} in the first reduced-pressure zone 170.

In addition to the features described above in relation to the first amplifier chamber 130, the amplification apparatus 200 additionally comprises a second amplifier chamber 230 upstream of the first amplifier chamber 130, the second amplifier chamber comprising a second amplifier inlet 290, multiple second primary inlets 220, and a second outlet 190 which forms the first amplifier inlet 190 of the first amplifier chamber 130.

The structure and function of the second amplifier chamber 230 and its respective inlets and outlets is substantially as described above in relation to the first amplifier chamber 130.

In the embodiment of FIG. 12A, the second amplifier inlet 290 is positioned in the back wall of the second amplifier chamber 230, in a position that is downstream of the second primary inlets 220. In the embodiment of FIG. 12B, the second amplifier inlet 290 is positioned further downstream in the second amplifier chamber 230, roughly half way between the second primary inlets 220 and the second outlet 190. In both embodiments, the second outlet/first inlet 190 is positioned well downstream of the first primary inlets 120.

In use, the embodiments of FIGS. 12A and 12B operate on the same principles described above for single-stage cyclonic amplifiers. The first amplifier chamber 130 generates a first low pressure zone 170 having a negative pressure P_{Z1} in response to a negative pressure Pₒᵤₜ being applied to the apparatus outlet 155. The negative pressure P_{Z1} then drives the second amplifier chamber 230 by drawing the second primary airflow through the second primary inlets 220 and downstream through the second amplifier chamber 230 as a swirling cyclonic primary flow. This second primary flow creates a second reduced-pressure zone 270 having a negative pressure P_{Z2} on the apparatus axis in the second amplifier chamber 230. This negative pressure P_{Z2} acts to draw a second flow of air into the apparatus through the inlet conduit 280 and through the second amplifier inlet 290. The second flow of air is injected into the low pressure (P_{Z2}) core of the swirling second primary flow and combines with the second primary flow as it passes through the second outlet/first amplifier inlet 190, to be combined with the first primary flow before exiting the apparatus 200 out of the outlet 150.

In the embodiments of FIGS. 12A and 12B, |P_{Z2}|>|P_{Z1}|>|Pₒᵤₜ|>|Pₐₜₘ|, and P_{z2}<P_{Z1}<Pₒᵤₜ<Pₐₜₘ.

In a preferred embodiment, the first amplifier chamber 130 has a conical swirl chamber geometry that is designed so that a pressure drop across it of −4 kPa creates a flowrate through it of 26.5 LPM (litres per minute), then using the 1.6× amplification factor discussed earlier, a maximum (negative) core pressure P_{Z1} of −6.4 kPa can be achieved in the first reduced-pressure zone 170.

This amplified negative pressure is used to the second amplifier chamber 230, which may be a smaller swirl chamber that is designed to run at a flowrate of 10 LPM with a driving pressure of −6.4 kPa. When combined with the first amplifier chamber 130, in a nested and coaxial manner, the core pressure P_{Z1} of the first amplifier chamber drives the smaller second amplifier chamber at a flowrate of 10 LPM. Using the pressure amplification factor of 1.6× as before, the new core pressure is now −10.2 kPa, and the total (combined) flowrate through the two amplifiers is 36.5 LPM.

Like the embodiments of FIGS. 8C and 8D, the embodiments of FIGS. 12C and 12D illustrate an inhaler apparatus which includes a deagglomerator 195. The deagglomerator is positioned upstream of the most-upstream amplifier inlet, which in this case is the second amplifier inlet 290. These embodiments function like those of FIGS. 12A and 12B, except that the incoming second fluid flow passes through the deagglomerator 195 and entrains a powdered medicament before reaching the second amplifier inlet 290.

In the embodiments of FIGS. 12C and 12D: |P_{Z2}|>|P_{Z1}|>|P_{D}|>|Pₒᵤₜ|>|Pₐₜₘ|; P_{Z2}<P_{D}<P_{Z1}<Pₒᵤₜ<Pₐₜₘ; Q_{b1}>Q_{b2}>Q_{D}; Qₒᵤₜ=Q_{b1}+Q_{b2}+Q_{D}. It is desirable to have P_{D} as close to P_{Z2} as possible.

### Two Stage Amplification Apparatus: First Cyclone Amplifier and Second Sheath Flow Amplifier

FIGS. 13A to 13F illustrate various embodiments of a two-stage amplification apparatus 300 according to the present invention, in which the second amplifier is a non-cyclonic amplifier.

Like the embodiments of FIGS. 12A-12D, in the two-stage amplification apparatus 300 of FIGS. 13A-13F a second amplifier chamber 330 is arranged upstream of, and nested inside the inlet end of, the first amplifier chamber 130, so the first amplifier chamber remains the most-downstream amplifier chamber in the apparatus.

As shown in FIGS. 13A-13F, a frusto-conical second amplifier chamber 330 is added in series with the first amplifier chamber 130. The second amplifier chamber 330 is arranged upstream of the first amplifier so that, in use, the first reduced-pressure zone 170 in the first amplifier chamber 130 drives a second primary fluid flow in the second amplifier chamber 330, which in turn establishes a second reduced-pressure zone 370 having a larger negative pressure than the negative pressure in the first reduced-pressure zone 170.

Unlike the cyclonic embodiments of FIGS. 12A-12D, the second amplifier chamber 330 is a non-cyclonic second amplifier chamber 330. The second amplifier chamber 330 is instead configured to generate a non-cyclonic swirling flow characterised by a swirl number S of less than 0.5, which indicates that the axial component of the flow dominates over its tangential component.

In FIGS. 13A, and 13C-13F, the second amplifier chambers 330 are configured to create a non-swirling sheath flow as the second primary airflow. The swirl number of the second primary flow in these devices is therefore very low, for example less than 0.2 or less than 0.1, preferably less than 0.05.

In addition to the features described above in relation to the first amplifier chamber 130, the amplification apparatus 300 comprises a second amplifier chamber 330 upstream of the first amplifier chamber 130, the second amplifier chamber comprising a second amplifier inlet 390 on the central axis of the apparatus, and a second primary inlet 320 surrounding the second amplifier inlet 390. The frusto-conical second amplifier chamber 330 narrows from a wide upstream end at the second primary inlet 320, to a second outlet 190 at the narrow end of the chamber. The second outlet forms the first amplifier inlet 190 of the first amplifier chamber 130.

The second amplifier inlet 390 is formed by the open downstream end of an inlet conduit 380. The upstream end of the inlet conduit 380 is couplable to, or may comprise, a deagglomerator 195 such as a blister filled with powdered medicament.

The second primary inlet 320 is formed by the open inlet end of the second amplifier chamber 330, and extends coaxially around the inlet conduit 380, so that air drawn into the apparatus through the second primary inlet 320 flows parallel to the central axis of the device.

The second amplifier chamber 330 may simply be a frusto-conical conduit through which the second primary flow passes as a sheath flow. Alternatively, one or more straight radial vanes 335 may be positioned in the second amplifier chamber 330. The straight vanes 335 may help to direct the second primary flow through the apparatus as a sheath (i.e. non-swirling) flow.

In FIG. 13B, the second amplifier chamber 330 includes a pair of angled vanes 345 which extend helically around the inlet conduit 380. The angled vanes 345 are configured to impart a slight swirl to the second primary airflow as it flows downstream through the second amplifier chamber 330. In order to ensure that the swirl flow is only slightly swirling, and does not establish a strongly swirling cyclonic flow, the angle of the angled vanes 345 relative to the central axis of the second amplifier chamber is restricted to a low angle, such as 10 degrees or 5 degrees. This ensures that the second primary flow has a swirl number of less than 0.5, preferably less than 0.4 or 0.3.

As shown in FIGS. 13A, 13B and 13E, the second outlet/first amplifier inlet 190 is positioned just upstream of the narrowest portion of the first amplifier chamber 130, and the second amplifier inlet 390 is positioned just upstream of the second outlet 190. The second outlet 190 constitutes the narrowest portion of the second amplifier chamber. An adjustable apparatus used to investigate the relative positions of these apertures is shown in FIG. 13E.

FIGS. 13C and 13D illustrate alternative embodiments in which the inlet conduit 380 extends through the second outlet 190, so that the second amplifier inlet 390 is actually positioned in the first amplifier chamber 130. Whilst these embodiments provide amplification, they exhibit poorer amplification than those in which the second amplifier inlet 390 is level with or upstream of the second outlet 190. Thus in some embodiments of the present invention, the second amplifier inlet may be positioned radially within, and downstream of, the first amplifier inlet. For example the inlet conduit may extend axially through the entire second amplifier chamber, and the second amplifier inlet may be positioned downstream of the second outlet, in the first amplifier chamber.

The inventors have found that the negative amplification performance of the apparatus 300 can be maximised by arranging the second amplifier inlet in the downstream end of the second amplifier chamber, and by positioning the first amplifier inlet in the downstream end of the first amplifier chamber, as this takes advantage of the venturi effect by virtue of the distance from the amplifier inlet to the surrounding chamber walls. This embodiment having a second sheath-flow amplifier advantageously increases performance relative to a single-stage cyclone amplifier while not having the complexity of two nested cyclones.

A deagglomerator 195 and apparatus inlet 105 are illustrated schematically in FIG. 13B, but all of the illustrated embodiments are intended to comprise, or to be suitable for connection to a deagglomerator upstream of the second amplifier inlet 390.

In use, the first amplifier chamber 130 in the embodiments of FIGS. 13A-13E operates on the same principles described above for single-stage cyclonic amplifiers. The first amplifier chamber 130 generates a first low pressure zone 170 having a negative pressure P_{Z1} in response to a negative pressure Pₒᵤₜ being applied to the apparatus outlet 155. The negative pressure P_{Z1} then drives the second amplifier chamber 330 by drawing the second primary airflow through the second primary inlet 320 and downstream through the second amplifier chamber 330 as a non-swirling sheath airflow (or a slightly swirling flow in the case of FIG. 13B). Due to the venturi effect and the narrowing shape of the second amplifier chamber 330, this “sheath flow” second primary flow creates a second reduced-pressure zone 370 having a negative pressure P_{Z2} on the apparatus axis in the second amplifier chamber 330. This negative pressure P_{Z2} acts to draw a second flow of air into the apparatus through the inlet conduit 380 and through the second amplifier inlet 390. The second flow of air is drawn into into the low pressure (P_{Z2}) core of the second primary flow (which is a sheath flow) and combines with the second primary flow as it passes through the second outlet/first amplifier inlet 190, to be combined with the first primary flow before exiting the apparatus 300 out of the outlet 150.

The inventors have found that two-stage amplification apparatuses containing a sheath flow second amplifier chamber provide better amplification than all single-stage amplification apparatuses, and better amplification than two-stage cyclonic apparatuses of the type shown in FIGS. 12A-12D. In one example, when a sheath flow second amplifier was added to a cyclonic first amplifier, the amplification performance was increased from an amplification of 1.36× to 2.28× in the two-stage amplifier.

FIG. 16 shows the sort of performance uplift that adding a sheath-flow brings to a single-stage cyclone: in FIGS. 16, A, B and C are all sheath-flow concepts, and these designs exhibit the highest levels of negative pressure amplification. All three designs containing a second “sheath-flow” amplifier stage created roughly 2× amplification between the apparatus outlet and the deagglomerator/blister. Interestingly, the performance uplift created by adding a second sheath-flow amplifier to a first cyclonic amplifier is greater than adding a second sheath-flow amplifier to a venturi (effectively making a 2-stage venturi).

### Two Stage Amplification Apparatus: First Sheath Flow Amplifier and Second Sheath Flow Amplifier

FIG. 14 illustrates an alternative two-stage amplifier apparatus 400, in which the first stage (downstream amplifier) is a first venturi amplifier chamber 410 and the second stage (upstream amplifier) is a second venturi amplifier chamber 420. Both of these chambers and their respective primary inlets 425, 435 are configured to generate non-swirling sheath flows therethrough as the first and second primary airflows. The second venturi amplifier chamber 420 also comprises a straight vane 445 to channel sheath flow air axially through the apparatus. As there is no cyclonic swirling flow to generate a low pressure core, the reduced-pressure zones in both amplifier chambers are established as a result of the venturi effect occurring where the airflows are constricted. In this embodiment, the first outlet 450 of the first venturi amplifier chamber 410 is formed by a narrowed neck portion of the downstream frusto-conical chamber, the first amplifier inlet/second amplifier outlet 460 is nested within the downstream amplifier chamber and positioned just upstream of the first outlet 450, and the second amplifier inlet 490 is nested within the upstream amplifier chamber and positioned just upstream of the second outlet/first amplifier inlet 460. The proximity of these apertures to one another advantageously maximises the venturi effect pressure-reduction experienced at the second amplifier inlet 490 to maximise the amplification produced by the device.

### Three Stage Cyclone Amplification Apparatuses

FIG. 15 illustrates a three-stage amplifier apparatus 500, in which all three amplifier chambers are cyclonic amplifier chambers. Like in the two-stage embodiments described above, the additional third amplification chamber 530 is added to the upstream end of the apparatus, and increases the overall pressure drop across the apparatus in a stepwise manner. The illustrated embodiment results in a total pressure amplification of just over 4×. Adding further amplifier stages to the apparatus reduces the flowrate available for the deagglomerator, so negative pressure amplification is increased, albeit at a reduced deagglomerator flowrate.

The negative pressure amplification apparatus of FIG. 15 contains all of the features of FIG. 12C, in addition to a third cyclonic amplifier chamber 530 positioned upstream of the second amplifier chamber 230 and downstream of the deagglomeration chamber 195.

The deagglomeration chamber, or “engine”, is in fluid communication with the third amplifier chamber 530 via the amplifier inlet 590, which is positioned in the third reduced-pressure zone 570 inside the third amplifier chamber. The negative pressure P_{Z3} in the third reduced-pressure zone draws air into the deagglomeration engine 195 through the apparatus inlet 105, and downstream through the inlet conduit 580 into the third amplifier chamber 530.

### Performance Comparison

FIG. 16 compares the negative pressure amplification achieved by six different designs of amplification apparatus in response to an outlet negative pressure Pₒᵤₜ of 4 kPa. P_{b} is the negative pressure experienced in the blister (deagglomerator) which is upstream of the most-upstream amplifier inlet.

HF, CS and HiRes represent the performance of the amplification apparatus when connected to three different upstream deagglomerators, each with a different flow resistance. The HF deagglomerator has the lowest resistance, the CS deagglomerator has a medium flow resistance, while the HiRes deagglomerator has the highest resistance.

The tested embodiments are:
A—2-stage amplifier: first stage cyclone amplifier, second stage sheath flow amplifier
B—2-stage amplifier: first stage cyclone amplifier, second stage sheath flow amplifier
C—2-stage amplifier: first stage cyclone amplifier, second stage sheath flow amplifier
D—Two-stage venturi amplifier
E—Single-stage venturi amplifier
F—Single-stage swirl/cyclone amplifier

There was little difference in performance between the single stage swirl amplifier F and the single-stage venturi amplifier E. Adding a ‘sheath flow’ upstream amplifier to the venturi to create a 2-stage venturi amplifier D led to a performance uplift, but the greatest improvement in performance was shown in designs A-C as a result of adding a sheath flow amplifier upstream of a single-stage swirl amplifier.

FIG. 17 illustrates the negative pressure amplification and additional power performance of the particularly preferred negative pressure amplification apparatus shown in FIG. 13A, which contains a first (downstream) cyclonic amplifier chamber, and a second (upstream) sheath flow amplifier chamber. This amplifier is able to convert the 4 kPa supplied at the apparatus outlet by the patient to a negative pressure of ˜9.5 kPa for the deagglomeration engine, at a flowrate through the deagglomeration engine of ˜3 LPM. The flowrate at the outlet is ˜53 lpm and the outlet pressure is ˜4 kPa. Moreover, before the blister (deagglomeration engine) is pierced there is no flow through it, so it effectively has infinite resistance. With infinite resistance the amplification is closer to 4×—so if a breath-actuated mechanism (BAM) is set to actuate at 1.5 kPa then the pressure drop available to actuate the BAM is over 5 kPa, which provides substantially more motive force to pierce the blister.

### PREFERRED ASPECTS

Preferred aspects of the invention are set out below by way of the following numbered clauses:
1. A negative pressure amplifier apparatus, comprising:
   a first amplifier chamber comprising a first amplifier inlet, a first primary inlet, and a first outlet, in which the first outlet forms, or is in fluid connection with, an apparatus outlet;
   and
   a second amplifier chamber upstream of the first amplifier chamber, the second amplifier chamber comprising a second amplifier inlet, a second primary inlet, and a second outlet which forms, or is in fluid connection with, the first amplifier inlet of the first amplifier chamber;
   the first amplifier chamber being configured to establish, in response to fluid being drawn from the apparatus outlet, a first primary fluid flow from the first primary inlet to the first outlet, and to create a first reduced-pressure zone of fluid at the first amplifier inlet, and to establish a second primary fluid flow from the second primary inlet to the second outlet and into the first amplifier chamber through the first amplifier inlet, wherein a second reduced-pressure zone of fluid is created at the second amplifier inlet,
   in which the second amplifier chamber is configured so that the axial component of the second primary fluid flow is greater than the tangential component of the second primary fluid flow.
2. A negative pressure amplification apparatus according to clause 1, in which the first amplifier chamber is configured to generate a pressure drop between the first outlet and the first amplifier inlet, such that in response to fluid being drawn from the apparatus outlet by an outlet negative pressure, the first reduced-pressure zone of fluid at the first amplifier inlet experiences a first inlet negative pressure that is greater in magnitude than the outlet negative pressure, and to generate a pressure drop between the first reduced-pressure zone and the second amplifier inlet, such that in response to fluid being drawn from the second outlet by the first inlet negative pressure, the second reduced-pressure zone of fluid at the second amplifier inlet experiences a second inlet negative pressure that is greater in magnitude than the first inlet negative pressure.
3. A negative pressure amplification apparatus according to clause 1 or 2, in which the first amplifier chamber is frusto-conical, with the first outlet formed by the narrowest portion of the frusto-conical first amplifier chamber.
4. A negative pressure amplification apparatus according to clause 1, 2 or 3, in which the first amplifier inlet is positioned on the central axis of the first amplifier chamber closer to the first outlet than the first primary inlet.
5. A negative pressure amplification apparatus according to any preceding clause, in which the first amplifier chamber has a length defined by the distance along the central axis between a downstream-edge of the first primary inlet and the first outlet at the downstream end of the chamber, and in which the first amplifier inlet is positioned in the downstream 50% of the first amplifier chamber, preferably in the downstream-most 40%, or 30%, or 20%, or 10% of the first amplifier chamber.
6. A negative pressure amplification apparatus according to any preceding clause, in which the first amplifier chamber is a first cyclone chamber operable to establish a cyclonic first primary fluid flow between the first primary inlet and the first outlet in response to fluid being drawn from the first outlet, preferably in which the swirl number S of the first primary fluid flow, defined as the ratio of the axial flux of angular momentum to the axial flux of the axial momentum, is greater than 0.5.
7. A negative pressure amplifier apparatus according to clause 6, in which the first amplifier chamber is a uniflow frusto-conical swirl chamber, in which the first primary inlet comprises one or more tangential inlets which create a swirling first primary fluid flow within the first amplifier chamber.
8. A negative pressure amplifier apparatus according to any of clauses 1 to 5, in which the first primary inlet and the first amplifier chamber are configured to establish the first primary fluid flow as a sheath flow between the first primary inlet and the first outlet, preferably in which the swirl number S of the first primary fluid flow is less than 0.5, or less than 0.4, or less than 0.3, or less than 0.2.
9. A negative pressure amplifier apparatus according to any preceding clause, in which the second amplifier inlet is positioned on the central axis of the second chamber, in the second reduced-pressure zone, and/or in which the second amplifier inlet is in fluid communication with an apparatus inlet.
10. A negative pressure amplifier apparatus according to any preceding clause, in which the second amplifier chamber is frusto-conical, with the second outlet formed by the narrowest portion of the frusto-conical second chamber.
11. A negative pressure amplifier apparatus according to any preceding clause, in which the first and second amplifier chambers are both frusto-conical and aligned coaxially along a central axis.
12. A negative pressure amplifier apparatus according to clause 10 or 11, in which the outlet end of the second frusto-conical chamber is nested within the inlet end of the first frusto-conical chamber, with the first amplifier inlet positioned downstream of the first primary inlet.
13. A negative pressure amplifier apparatus according to any preceding clause, in which the second amplifier chamber has a length defined by the distance along the central axis between a downstream edge of the second primary inlet and the second outlet at the downstream end of the second amplifier chamber, and in which the second amplifier inlet is positioned in the downstream 50% of the second amplifier chamber, preferably in the downstream-most 40%, or 30%, or 20%, or 10% of the second amplifier chamber.
14. A negative pressure amplifier apparatus according to any preceding clause, in which the second amplifier inlet is narrower than the second outlet and the first outlet.
15. A negative pressure amplifier apparatus according to any preceding clause, in which the second primary inlet and the second amplifier chamber are configured to establish a second primary fluid flow as a sheath flow between the second primary inlet and the second outlet, preferably in which the swirl number S of the second primary fluid flow is less than 0.5, or less than 0.4, or less than 0.3, or less than 0.2, or less than 0.1.
16. A negative pressure amplifier apparatus according to any preceding clause, in which the second amplifier chamber comprises one or more vanes configured to direct the second primary fluid flow towards the second outlet.
17. A negative pressure amplifier apparatus according to clause 16, in which the one or more vanes are aligned parallel with the central axis of the second amplifier chamber.
18. A negative pressure amplifier apparatus according to clause 16, in which the one or more vanes are angled at 25 degrees or less relative to the central axis of the second amplifier chamber, or 20 degrees or less, or 15 degrees or less, or 10 degrees or less relative to the central axis of the second amplifier chamber, preferably between 2.5 and 7.5 degrees relative to the central axis of the second amplifier chamber.
19. A negative pressure amplifier apparatus according to any preceding clause, in which the second primary inlet is configured to direct fluid into the second amplifier chamber in an axial direction, so that the fluid flow is directed along the central axis of the chamber towards the second outlet.
20. A negative pressure amplifier apparatus according to any preceding clause, in which the second primary inlet surrounds the second amplifier inlet, or in which a plurality of second primary inlets are arranged around the second amplifier inlet.
21. A negative pressure amplifier apparatus according to any preceding clause, in which the second primary fluid flow creates the second reduced-pressure zone of fluid at the second amplifier inlet, such that a second fluid flow is drawn through the second amplifier inlet into the second reduced-pressure zone.
22. A negative pressure amplifier apparatus according to clause 21, in which the second amplifier inlet is positioned downstream of the second primary inlet, such that in use, the second fluid flow is introduced to the second reduced-pressure zone downstream of the second primary inlet.
23. A negative pressure amplifier apparatus according to clause 21 or 22, in which the second amplifier inlet is configured so that the second fluid flow is a non-swirling fluid flow with a swirl number of less than 0.2, or less than 0.1, at the second amplifier inlet.
24. A negative pressure amplifier apparatus according to clause 21, 22 or 23, in which the second primary fluid flow has a swirl number S which falls between the swirl number of the second fluid flow and the swirl number of the first primary flow.
25. A negative pressure amplifier apparatus according to clause 24, in which the first primary flow is a cyclonic flow with a swirl number greater than 0.5 at the first outlet, the second fluid flow is a non-swirling fluid flow with a swirl number of less than 0.2, or 0.1 at the second amplifier inlet, and the second primary flow is a sheath flow which has a swirl number at the second outlet which is greater than that of the second fluid flow, but less than that of the first primary flow.
26. An inhaler apparatus, comprising a negative pressure amplification apparatus according to any preceding clause, in which the inhaler comprises or is couplable to a source of medicament such that in response to fluid being drawn from the apparatus outlet by an outlet negative pressure, the medicament is entrained in a fluid flow upstream of the or each amplifier chamber and delivered into the most-upstream amplifier chamber through the amplifier inlet.
27. An inhaler apparatus according to clause 26, in which the medicament is a dry powdered medicament, and the inhaler is a dry powder inhaler.
28. An inhaler apparatus according to clause 26 or 27, in which the inhaler comprises a deagglomeration chamber containing the source of medicament, or positioned between the source of medicament and the amplifier inlet of the most-upstream amplifier inlet, preferably in which the deagglomeration engine is a blister.
29. An inhaler apparatus according to clause 26, 27 or 28, in which the inhaler comprises an inhaler inlet configured to direct a flow of fluid into contact with the source of medicament, and one or more primary inlets configured to direct a flow of fluid into the primary inlets of the or each amplifier chamber.
30. An inhaler apparatus according to any of clauses 26 to 29, in which the inhaler comprises a mouthpiece surrounding the first outlet.

1. A negative pressure amplifier apparatus, comprising:
a first amplifier chamber comprising a first amplifier inlet, a first primary inlet, and a first outlet;
in which the first outlet forms, or is in fluid connection with, an apparatus outlet;
the first amplifier chamber being configured to establish, in response to fluid being drawn from the apparatus outlet, a first primary fluid flow from the first primary inlet to the first outlet, and to create a first reduced-pressure zone of fluid at the first amplifier inlet, such that a first fluid flow is drawn through the first amplifier inlet into the first reduced-pressure zone,
in which the first amplifier inlet is positioned downstream of the first primary inlet, such that in use, the first fluid flow is introduced to the first reduced-pressure zone downstream of the first primary inlet.

2. A negative pressure amplification apparatus according to claim 1, in which the first amplifier chamber is configured to generate a pressure drop between the first outlet and the first amplifier inlet, such that in response to fluid being drawn from the apparatus outlet by an outlet negative pressure, the first reduced-pressure zone of fluid at the first amplifier inlet experiences a first inlet negative pressure that is greater in magnitude than the outlet negative pressure.

3. A negative pressure amplification apparatus according to claim 1 or 2, in which the first amplifier chamber is frusto-conical, with the first outlet formed by the narrowest portion of the frusto-conical first amplifier chamber.

4. A negative pressure amplification apparatus according to claim 1, 2 or 3, in which the first amplifier inlet is positioned on the central axis of the first amplifier chamber closer to the first outlet than the first primary inlet.

5. A negative pressure amplification apparatus according to any preceding claim, in which the first amplifier inlet is in fluid communication with an apparatus inlet.

6. A negative pressure amplification apparatus according to any preceding claim, in which the first amplifier chamber has a length defined by the distance along the central axis between a downstream-edge of the first primary inlet and the first outlet at the downstream end of the chamber, and in which the first amplifier inlet is positioned in the downstream 50% of the first amplifier chamber, preferably in the downstream-most 40%, or 30%, or 20%, or 10% of the first amplifier chamber.

7. A negative pressure amplification apparatus according to any preceding claim, in which the first amplifier chamber is a first cyclone chamber operable to establish a cyclonic first primary fluid flow between the first primary inlet and the first outlet in response to fluid being drawn from the first outlet, preferably in which the swirl number S of the first primary fluid flow, defined as the ratio of the axial flux of angular momentum to the axial flux of the axial momentum, is greater than 0.5.

8. A negative pressure amplifier apparatus according to claim 7, in which the first amplifier chamber is a uniflow frusto-conical swirl chamber, in which the first primary inlet comprises one or more tangential inlets which create a swirling first primary fluid flow within the first amplifier chamber.

9. A negative pressure amplifier apparatus according to any of claims 1 to 6, in which the first primary inlet and the first amplifier chamber are configured to establish the first primary fluid flow as a sheath flow between the first primary inlet and the first outlet, preferably in which the swirl number S of the first primary fluid flow is less than 0.5, or less than 0.4, or less than 0.3, or less than 0.2.

10. A negative pressure amplifier apparatus according to any preceding claim, additionally comprising a second amplifier chamber upstream of the first amplifier chamber, the second amplifier chamber comprising a second amplifier inlet, a second primary inlet, and a second outlet which forms, or is in fluid connection with, the first amplifier inlet of the first amplifier chamber,
in which the second amplifier chamber is configured to establish, in response to fluid being drawn from the apparatus outlet, a second primary fluid flow from the second amplifier inlet through the second amplifier chamber and through the second outlet into the first amplifier chamber, and to create a second reduced-pressure zone of fluid at the second amplifier inlet.

11. A negative pressure amplifier apparatus according to claim 10 and claim 2, in which the second amplifier chamber is configured to generate a pressure drop between the second outlet and the second amplifier inlet, such that in response to fluid being drawn from the second outlet by the first inlet negative pressure, a second inlet negative pressure is generated at the second amplifier inlet in the second reduced-pressure zone of fluid, the second inlet negative pressure being greater in magnitude than the first inlet negative pressure.

12. A negative pressure amplifier apparatus according to claim 10 or 11, in which the second amplifier inlet is positioned on the central axis of the second chamber, in the second reduced-pressure zone.

13. A negative pressure amplifier apparatus according to claim 10, 11 or 12, in which the second amplifier chamber is frusto-conical, with the second outlet formed by the narrowest portion of the frusto-conical second chamber.

14. A negative pressure amplifier apparatus according to any of claims 10 to 13, in which the first and second amplifier chambers are both frusto-conical and aligned coaxially along a central axis.

15. A negative pressure amplifier apparatus according to any of claims 10 to 14, in which the outlet end of the second frusto-conical chamber is nested within the inlet end of the first frusto-conical chamber, with the first amplifier inlet positioned downstream of the first primary inlet.

16. A negative pressure amplifier apparatus according to any of claims 10 to 15, in which the second amplifier chamber has a length defined by the distance along the central axis between a downstream edge of the second primary inlet and the second outlet at the downstream end of the second amplifier chamber, and in which the second amplifier inlet is positioned in the downstream 50% of the second amplifier chamber, preferably in the downstream-most 40%, or 30%, or 20%, or 10% of the second amplifier chamber.

17. A negative pressure amplifier apparatus according to any of claims 10 to 16, in which the second amplifier inlet is narrower than the second outlet and the first outlet.

18. A negative pressure amplifier apparatus according to any of claims 10 to 17, in which the second primary inlet and the second amplifier chamber are configured to establish a second primary fluid flow as a sheath flow between the second primary inlet and the second outlet, preferably in which the swirl number S of the second primary fluid flow is less than 0.5, or less than 0.4, or less than 0.3, or less than 0.2.

19. A negative pressure amplifier apparatus according to claim 18, in which the second amplifier chamber comprises one or more vanes configured to direct the second primary fluid flow towards the second outlet.

20. A negative pressure amplifier apparatus according to any of claims 10 to 19, in which the second amplifier chamber is a second cyclone chamber operable to establish a second cyclonic primary fluid flow between the second primary inlet and the second outlet in response to fluid being drawn from the second outlet, preferably in which the swirl number S of the second primary fluid flow is greater than 0.5.

21. A negative pressure amplifier apparatus according to claim 20, in which the second amplifier chamber is a uniflow frusto-conical swirl chamber, in which the second primary inlet comprises one or more tangential inlets which are configured to create a swirling flow within the second amplifier chamber.

22. A negative pressure amplifier apparatus according to any of claims 10 to 21, in which at least one of the first amplifier chamber and the second amplifier chamber is a cyclone chamber operable to establish a cyclonic primary fluid flow through that amplifier chamber in response to fluid being drawn from the apparatus outlet, preferably in which the swirl number S of the primary fluid flow through that amplifier is greater than 0.5.

23. A negative pressure amplifier apparatus according to claim 22, in which at least one of the first amplifier chamber and the second amplifier chamber is a uniflow frusto-conical swirl chamber, in which the primary inlet to that amplifier chamber comprises one or more tangential inlets which are configured to create a swirling flow within that amplifier chamber.

24. A negative pressure amplifier apparatus according to any of claims 10 to 23, in which the second primary fluid flow creates the second reduced-pressure zone of fluid at the second amplifier inlet, such that a second fluid flow is drawn through the second amplifier inlet into the second reduced-pressure zone.

25. A negative pressure amplifier apparatus according to claim 24, in which the second amplifier inlet is positioned downstream of the second primary inlet, such that in use, the second fluid flow is introduced to the second reduced-pressure zone downstream of the second primary inlet.

26. A negative pressure amplifier apparatus according to claim 24 or 25, in which the second amplifier inlet is configured so that the second fluid flow is a non-swirling fluid flow with a swirl number of less than 0.2, or less than 0.1, at the second amplifier inlet.

27. A negative pressure amplifier apparatus according to clause 24, 25 or 26, in which the second primary fluid flow has a swirl number S which falls between the swirl number of the second fluid flow and the swirl number of the first primary flow.

28. A negative pressure amplifier apparatus according to clause 27, in which the first primary flow is a cyclonic flow with a swirl number greater than 0.5 at the first outlet, the second fluid flow is a non-swirling fluid flow with a swirl number of less than 0.2, or 0.1 at the second amplifier inlet, and the second primary flow is a sheath flow which has a swirl number at the second outlet which is greater than that of the second fluid flow, but less than that of the first primary flow.

29. An inhaler apparatus, comprising a negative pressure amplification apparatus according to any preceding claim, in which the inhaler comprises or is couplable to a source of medicament such that in response to fluid being drawn from the apparatus outlet by an outlet negative pressure, the medicament is entrained in a fluid flow upstream of the or each amplifier chamber and delivered into the most-upstream amplifier chamber through the amplifier inlet.

30. An inhaler apparatus according to claim 29, in which the medicament is a dry powdered medicament, and the inhaler is a dry powder inhaler.

31. An inhaler apparatus according to claim 29 or 30, in which the inhaler comprises a deagglomeration chamber containing the source of medicament, or positioned between the source of medicament and the amplifier inlet of the most-upstream amplifier inlet, preferably in which the deagglomeration engine is a blister.

32. An inhaler apparatus according to claim 29, 30 or 31, in which the inhaler comprises an inhaler inlet configured to direct a flow of fluid into contact with the source of medicament, and one or more primary inlets configured to direct a flow of fluid into the primary inlets of the or each amplifier chamber.

33. An inhaler apparatus according to any of claims 29 to 32, in which the inhaler comprises a mouthpiece surrounding the first outlet.
